SEQUENCE LISTING (256 sequences)

SEQ ID NO: 1 - DNA, 200 bp, Homo sapiens
aagaatggaaggacagcttgctacttctgggtgttcatcatgagcagttaaggctcaccatatggtctccactgatatttgggggaaggagacttgttacyacctaataatgagatgaaagatcctgattccctacttggtttactatgacaccgccctggcatgatggttcaggaaactctttacatttacagattgtc

SEQ ID NO: 2 - DNA, 200 bp, Homo sapiens
tactgctgtctttggaacactgttactcatcctacaaggcctcaatcaagaatcttaggaggtcattggtgcctacctgcccatggcagagaccaccaacmccttctgagagctgccacagtgctgctttctaacatgtctacactagctcctcacagtgtgcggtgattgttgggtgaaagctctgtttctcatagtga

SEQ ID NO: 3 - DNA, 200 bp, Homo sapiens
tgatttgattttatccagtgttgcaaactgggtttcgtaagtgactcttttgtaaggagtttcagtgcaccccgtctgcaacccatgcatatcttatttamccccaagcttctaaggtagaatcctacttttttcaagtgctattgattagttgcctgtttagacttttgttatatgatttaaaattttaaaaatgataa

SEQ ID NO: 4 - DNA, 200 bp, Homo sapiens
gtgtttctaaagcaactccccaccaacctcttgggctgtggtgaccaagggctaaaaattaagtggggaccctggtgtctgacttttgctccacttctgayaccttctttctctcccgtttcactaggactggaaaatacaggcttttttcccaatccttgtttctctcaaaccaagacctcatctacataaaacatcta

SEQ ID NO: 5 - DNA, 200 bp, Homo sapiens
ccactgaaaatatcctctggtcatttatttggctcaacggacctatatggccattccctcttccacagttgtaaagactttgcctaatttctggcaactayagaagtagtggtaaggataatgtaaaatttctttttgtcttggaaattatcaaaccacttttgaagattgtattatctagtaatcagaaacttactttg

SEQ ID NO: 6 - DNA, 166 bp, Homo sapiens
taagggcttgagtatgcaaatatggaggctttctcaaatcacgtttcgtttaatttaacaccttcataaataggatggacttaagagatcatcccaacctycttactgtacagatcaagagatgggggtccggaaagctgctgatgttcatgctgtgagtttaaca

SEQ ID NO: 7 - DNA, 200 bp, Homo sapiens
tttgccaaccattcttcaccctgttgggagagaatacagaaatctgtattcaagcataaggtgctatacacttgtttgatgcctgcctctagggcaatgargaggtaagacaatctccccattacatgctttcgcagaggacagtctgtaaagttacagaactccattccttcttacgtaccttataccataatatacca

SEQ ID NO: 8 - DNA, 152 bp, Homo sapiens
aaggggcggcgctggccggactctctcgggtggactcccggagcggcggcggcggcggcggtggcagactgcgagtcacgtgcgacagaggaggcggagcrcggcggccgggtgggtggagaaaacaaagcccccagctgtcagcagaggaa

SEQ ID NO: 9 - DNA, 200 bp, Homo sapiens
attatataaatgaaactggtttaggatgtgtctttttttcccctctcttttaccttaactcaggttcaagcctttcttaagctctggtaacttgcatatayaacaaagttgaaataaaacagatttttatatagggaaagagagatgaggaagggaacatatccagttttctcactgtgaaacagagttagcaaggaagt

SEQ ID NO: 10 - DNA, 201 bp, Homo sapiens
gattatgttaacagaaattctcagcctgtgtgatttgtcatattttaatatttcaagccaccattgcagcttcttttaggtttggtttgtctgaggcaccrcagcgagtgcatgagcacacagactttgaatttgtataagtgaatggctcttccacttactttgtggctttgggctcatcacttaactcattgatataaa

SEQ ID NO: 11 - DNA, 200 bp, Homo sapiens
tgtgcagcaatagtaactggaacaacatttctgcgatgggcagaagtggcaaggattgtaactcattagactcagaaggcgaggtcacctgagtaagcagkgtccttagtggaacagtgggtgtaagaggaaggagagatgggtgggtgtggggcttcttgccatctacatatgtagggtctgccttttacttgcacatc

SEQ ID NO: 12 - DNA, 166 bp, Homo sapiens
gatgatacttagcttctgcgtcttcagtaaactcatctgtacaatggcctgttaaacctagctatttgaaaacatcagctaaaactaagtgatttttttgrtattcatttaggtcttttatttcttgatatgcttgaaatctaattgtcttttaaatgcactaata

SEQ ID NO: 13 - DNA, 151 bp, Homo sapiens
gattacaggcgtgagccacggcacctggagagaaaaaagcttctttaatgacgacctcaacataatttacatggagaagtttcttaggtggaaaaccgatytgtatggtcctcagaatgtaacctacaggttcttgccctcagaaaaatgc

SEQ ID NO: 14 - DNA, 200 bp, Homo sapiens
taaggaagagggacagctatgacctaatgcttgcttggaccactataagcatgccagggaaaatattcaggctaaattgtcggctctaagaacatgaagtrtattgatttctttattacagctagcagatatttaagaatgttagcacaggtctttgaataaattttgcttttaagagaagttactatttattcctaatt

SEQ ID NO: 15 - DNA, 200 bp, Homo sapiens
tggacaatgggaataaagcatttctaaagcaccgactggagaggaaggcaacagagacaaggagagaagccgagagacatgtctgcgtgctgccacgcatytgagcgattgctctgtgaagagttgtacactgaacactttcaggggaggctgtttacccaggcaatgtcctcaaacaagcctgtgccggggtgtcctgg

SEQ ID NO: 16 - DNA, 200 bp, Homo sapiens
agaggcggcggctccggaacgagctgctcccacagcccctccgctcctcgccgtctcgccttcgtccccagcttacccagttctgcgcgttattgctcagyttgactttcttgcacaggctcccggggatctccatgaccgcggagcgcgctgagcagccggggttctctgcgccggganggtagcaagaggagggcagg

SEQ ID NO: 17 - DNA, 200 bp, Homo sapiens
ttagcagtcccgcaaagcttgaggtttactcatttttactttctttagtctctgggcaaggaactaagaggtagtatagaattctagacgatggaaacttyagaatggacaggattttaaaaattatatattccattctcctgggttacaggtggtcttacagctaaactatttgagactgttgaagccattcgattttt

SEQ ID NO: 18 - DNA, 200 bp, Homo sapiens
ttatgaggaacagatgagatagtgaggttgagtgccagcacagtagccagtactcaatcaaatataactgtaaaacaaaggggcagttcctaaggcccaaratggagttttagtcaaaaggaaataaggcaaaaggccagttacaagatggagcaggccctgcagtggagttgaaaacccaggtcaccatgatgactgtg

SEQ ID NO: 19 - DNA, 200 bp, Homo sapiens
ttaggaaattttcaatggagaaaataagttccaaaagtcattctttctcagattaagatgtcttgggtttcagtttacttctttaaagaaagattcagagytagtactgaggacccagactttcttgatgtgggaaatagataattttctgttctgttgacattttttctctctcttctctcattttcaaagtacagttc

SEQ ID NO: 20 - DNA, 200 bp, Homo sapiens
taccatcctcacatcttgtatagactccttttatcagggcctggtggtcccagttcttcctagagaaacccatagcaacattcttgatggtcagtggtccrtggactgggggggtccccatggccatctgtccttcctcttggctcccttctgaggggcaggcagggtcctaaaaggacagagccctgtggacctggcag

SEQ ID NO: 21 - DNA, 173 bp, Homo sapiens
aaacattgtttacataagtaaacaagccagggctgctgtgaaagttattatggaagatacaatttatatattttctggaaaatatatcgcctcatatatayagacaaaacctttgctttaacatgtttgtgcaactaattacttactttccaatgagtacaatttcccactag

SEQ ID NO: 22 - DNA, 200 bp, Homo sapiens
gaggtgggcactggtggcttccagcctagggctgtgactctgctgttaggggcacgctgctacacaggcaccaacctctctagcacaggggttcctccaaktgtgctgtgggggcccccgagaccctttcaggggttcccccaggttccttgaaaggctccnctgattagatcaggcccaccaagatcacctccctttgg

SEQ ID NO: 23 - DNA, 201 bp, Homo sapiens
agagtcataagaagggttttctctctcctaaggaggaaccaaagggggaaaaaaagtctctttgcttctagacgttgtttctgtagatgtaaaggctgttmacttctgcagtcatcttgctgagaataaattcagaatgaagatggcaaaatggagcaagaaataagttaatgacccggaaggcctccttctgaagttgtt

SEQ ID NO: 24 - DNA, 200 bp, Homo sapiens
caccatttatttcctattttctctcctgagttaaataggaaacatgtctcgcattacttgaaaaatcaagtcaaactatgctcttactaggagttatggtyctttttatgtcttagatgatgcttgatctagatgaatgcggacttgctgtagctagataaatacaatgggagtttgaaggtgtttcgtagccctggaaa

SEQ ID NO: 25 - DNA, 200 bp, Homo sapiens
agaactgtggagcgattcctgatttttgagcaggaagagtgacaattcaaaacagtatttgactagattcacggctccgtagcatccccttgggtgggagsgggaaggctgactaggacctctgattcttctttccctgagctttgaaggctctgaaaatacagctggggggacttgcccagttttcttattaagcaatt

SEQ ID NO: 26 - DNA, 200 bp, Homo sapiens
agcacagttccaagatgcgctgacatgttctgcaggcgagagagcctttcgctggactgtgactcacggaggtggtgaggccgctggttctaaccaagagratgccagcgagaccactcagcctcccctggtggctgccctggtttaatgctcatgaaatgccagcctgtggccattctgctgggtaaagagggtggtgt

SEQ ID NO: 27 - DNA, 125 bp, Homo sapiens
cccagcaggaacccttctggaagggaatatttgcagagatttcccagatctatgtgcccttctgcttcctctatgcttactgctctcacctgggccacccmtgaaggtgggcttgctgtttcagc

SEQ ID NO: 28 - DNA, 200 bp, Homo sapiens
tggcgcgtgtgcaattagcaatgcagtgtatatacaagaagccatgctgttaacagtttatctcaagtaatttggtgtcatttacaaaaggaagaaattcytgccatcagaagggacagaaggagatggaatgatcaaatcacagagaaacactaaaattactaaatctacaacagctcgccttatttttcttggaccca

SEQ ID NO: 29 - DNA, 200 bp, Homo sapiens
gagacaacggggatcaggcagaggggtgggcacataggaggcaacgtaaaccagaactgaggctgggccaggccagggtgtcagaggctgaggcggggagkcgggctgcccttgggctggattgcaaggcgaagaagcaggagggagaggctgagagtgtggaggggagggggcttgtagcgtgtgattggagggaggac

SEQ ID NO: 30 - DNA, 200 bp, Homo sapiens
ctacccacaaaagtttctttgtacttatttacnagtcctgatgtttttgttagaaaaacattgccacctntttttacccagccaggtctcagcctaagtgycactgggtccagtttctctcatcttctgagcagttggcctaaagtgaccctatgttttccttttttctttctcctcttcctcttccacaccagtgcaaa

SEQ ID NO: 31 - DNA, 200 bp, Homo sapiens
tttttaggcttaaaaagatattttaatgtaactctatccaattctcaattttaggcctgccagaattgaaactcaagcttaactataatcatcaccaagcrttacttttgaagccctcccgtaaatatgaggctgttaaggatgcaatactataccgcgtgtctcttaggttagttggaaaaatgagttagaaaatgagg

SEQ ID NO: 32 - DNA, 200 bp, Homo sapiens
aaagctgagtaagcacctatagtaacaggtaacaaagtattctagggatcagctgctgtgagttagttcacaagtgctttaagtacgtgttaatcaaagaygatgtatttttcatttctgacaataaacatctggggcatcctacatttttcctgggcaatttgcctttatcattatattttaatctctgatgcaatctt

SEQ ID NO: 33 - DNA, 200 bp, Homo sapiens
ccacttccctctcacacagaatagatttccatagggtgagaattcaagagaaactttttaattgtacagcaaagtgtttactttgccaccaacctgccctrtagcttaagcatatggctaccttttctatcaacagctgggcttctgtaggtttggcactcggtataccatcccagcgcagtcaaaaacctgcaggtgac

SEQ ID NO: 34 - DNA, 200 bp, Homo sapiens
atgttgttcagagggctattcagagaatggaaacccaaggctcacctttcttgagtctaagtacccactcagccaagtatatttagcaaaatagaaaaacyaaactgctttggtgactggactttgccaatgtctgaaagaaagaagtaacagaaaggagagtctaaagttgatcctgtacagtataaaacatttcccca

SEQ ID NO: 35 - DNA, 200 bp, Homo sapiens
tggatgtactcatagagagggctggagcgcactgggacactggcaaactgatgagtgccgggctccagaagccgcccatggcccttcccttcttgggattktatctccccgacttggcggtgcctctgttcctccttctgctggcggcggatgtgttcttcccgtaatttcctttcccgttcctggcgacgtttctgctc

SEQ ID NO: 36 - DNA, 200 bp, Homo sapiens
gggaataaattagttttccttgacagtatatactgcctgtttctgtccatcctcttatttgcttctttttctttgctcttccactccagtccttaagcgaktttagttttattcgttcctcttgtcccttttcatgataacaatgtgtcatagtctgctttaaagtttttcatacttaccgatgttagttttattgctag

SEQ ID NO: 37 - DNA, 200 bp, Homo sapiens
tggggacagagcctgtggggagtcttgatgtagtcttgtccttgaaatccattcacggctctgctgagatctcgggaaagagtctctttataaatatgggkttttgttttttcaaattttggaaaagtcacctctggttttaaaacaattacttcccgctccaaacagattagtcacttcccaagaaccatgcttctctc

SEQ ID NO: 38 - DNA, 200 bp, Homo sapiens
attatagacaagcagagagggaatggatccgagggtgacctggctgaattaaaagtactgcatctaagtgtgtgcaggttgtagtccacagtaatggtgcyattcggagctgtgtgtacctgcactactgcatcaggtgaccttatccagcataacggtacaacagaaatttaacttgaaacaggtttattgacttgctt

SEQ ID NO: 39 - DNA, 201 bp, Homo sapiens
gtgacaaacctgagactagaaaactactacatatgacatcctacacactgactcccttcttcagctctgtctgataaagggatgagagatcgtttcttatyattacacatacacgccacttccgttccttccatctaaacctagttcagaaacaccgctaagaattcccacccccaaaaaaatggggatgggggaagagaa

SEQ ID NO: 40 - DNA, 200 bp, Homo sapiens
actgaggttagtctcttgtattaaactcttcacaaaatctgtttagcagcagcctttaatgcatctagattatggagcttttttccttaatccagctgatsagttacagcctgttagtaacatgaggggacattttggtgagaaatgggacttaactccttccagtgtccttagaacattttaattcatcccaactgtct

SEQ ID NO: 41 - DNA, 200 bp, Homo sapiens
cgagactcaagataatgtactaaagacctttagcacaatgtctgacatctggtgttcaataagtggtgatattgctcaatttcattccaattcaaacttayaaacatccatgggaagtctgctttatagacaagcaataggggagcacagtaattatttgggaggggaaatctcagcagttcttccaagtgacacattct

SEQ ID NO: 42 - DNA, 200 bp, Homo sapiens
atttgctgactttgccaagcgatgagccacacaggattgggaagcctgaatgtcatcagtcaaggcccagagtgctgaacctatgaggcctggggagaccrctgtgaattcctgcatcgtcactgagatcatcatatcctgtgaaaaacatctgccagccactgtctgtgttgtagaaagctgctgatgggaaaacttgg

SEQ ID NO: 43 - DNA, 200 bp, Homo sapiens
cataaaccctcaataaacctaagctactattattaagtactgtgtgtccacacaaaaaaaaggtctggaaagatatagccaccaggagtagttatgaggartgaggcagagaggctgatggaggacttccattttctattttgtatactacttttctagtgtttacaaatgagcttgtattgtttttatcatcagcaaaa

SEQ ID NO: 44 - DNA, 200 bp, Homo sapiens
atagcaggccagggctctgtaggcttgttttgctgctgtgcctgttgccaaatgcttgaaaatcctgaagtacaaacataacagtctgaattaaatttcaytgattttatctttctcttccttttctcactgtggtgcagttgttaatggaattctagccaaaaggatgtgggaggagaggaaggagccacaaagggaga

SEQ ID NO: 45 - DNA, 200 bp, Homo sapiens
ttagtcatgatgttctggtttttatttatacattcatctgaaagagcaaagtggtagagctaaaatcagtaagcttggctctgtttccaaactcataaaartgcaatatcgtggcagaaagattcttgtcctatttcttattttacctgggcttttgggatttcacatggacatagttgcaaaataatactccccaaact

SEQ ID NO: 46 - DNA, 200 bp, Homo sapiens
ctaaccaagcttgcccatatttcacatgttgagtttgttcattttaactgtcaattggcctaattttcccatttctagcattttaatttctccagagcttkcatactacattgaacaccaattcagtgacctggaacaattccactcgcatttgttccttttagaccatttcactgggagtatgtttccaatccacttat

SEQ ID NO: 47 - DNA, 200 bp, Homo sapiens
cagagacctggtcctaaatctgtgtgacttttgcgaaatccttttgcctcacagagctctaggttcttatctgtcaaacaggaaccagaccatcagatccrtagagatccaaccaatgaaggagacttcgaaaacttcacattgccgtgtcaatgtgaggttgctggatggttatgacaagaaatcacattaaattcact

SEQ ID NO: 48 - DNA, 200 bp, Homo sapiens
gcaggaggaagggtgcctgattcacaggggagatgcctgatggcgggtggggggctgctgttggcatccccactggaacgtttgtcctaatgggggcctgrctccctcatcagtggcttgtcccaaaacaacaaccagtgttcccaaacctatacttcctctttggatttttgtttttcatctagttggagggaataaaa

SEQ ID NO: 49 - DNA, 200 bp, Homo sapiens
tcattagtttattgcatccccaaggctttgataacaaatgaagcaaatattttcaaatttttctggttacccaataagaaaaatcatgtgactttccacayctgccatcattggaagtgacaaaactctagatacttgggtcattgttatcatttgtctatcttccttggtatgctctattcttcgttgtggagtaataa

SEQ ID NO: 50 - DNA, 200 bp, Homo sapiens
ccagggtcggcgcctgccccagcctgagtattgccctgattctctgtaccaagtgatgcagcaatgctgggaggcagacccagcagtgcgacccaccttcrgagtactagtgggggaggtggagcagatagtgtctgcactgcttggggaccattatgtgcagctgccagcaacctacatgaacttgggccccagcacct

SEQ ID NO: 51 - DNA, 200 bp, Homo sapiens
tctgacattctctggttataaaactagagtatcaaaatgcatgagagtgcctgctcagataaccatcagatgaatatgaagcttgctcatccctgggagtrcagaaatcagctccttttacctcttgaagaagtggatacagcatcctctactgctcaattgcataaaccagcacttgtccttggtgagccgttgtttgc

SEQ ID NO: 52 - DNA, 200 bp, Homo sapiens
atagaatttttggtatactgaacaaagggattaaaaaggtcagttttatgtagggactttctttgctggggagggaaggtctcatccccaagaattatttmtttattttcccttcccagagtttttacatggaagtatgaaatattaccttcctactatataattaatagaaaaaataaacagagcccagattatactat

SEQ ID NO: 53 - DNA, 200 bp, Homo sapiens
gcatagtactcagactcattgaaagtttatagaatttggcctgtgtggaaaactctgtgctccaagtacaacaactaactgaattctctaatttaaacaastttgaagggaagtgaaaggttataaaatgatacagactcataccgcagaatcaccttaaaatgcagctgttggagaaaaaaaaaaaggaagctttatgc

SEQ ID NO: 54 - DNA, 176 bp, Homo sapiens
ttttcctgggcttcatgggccatttaaattgtgaacccccagggacacagtgcttagcccaagccatcagtgcttgaaaataggcatttctgtcatcgagwttctgtatgttgagatatattttgagataggcttaagggtgaggacatttggctggagtcagcaataagttgcaa

SEQ ID NO: 55 - DNA, 201 bp, Homo sapiens
cacaagactagattgcaggaagatgtgaaagcatttggctaattctgacatttggctaataacatcctcacagtatgtcccaagtatcttctgcccttccyaccacggtgagcagtcatgacagtgagggtggcataggtgggatgtgtggctggcagaagccagtgggcaagagttgtccacagaatagtccatgagctt

SEQ ID NO: 56 - DNA, 200 bp, Homo sapiens
tagagggggtgcgggacggggactcacaggagatgcaggacggcccgaacatagtaattcctggtaaagggcccgaacagcttcaccacggcggtcatgtrcttctgtcccctgggggagggaggaaggcgagacggcgcggctgggcctctcccactcgggactcctttgctgccctgctgaccaccccagggcaccca

SEQ ID NO: 57 - DNA, 201 bp, Homo sapiens
gttcacttgctgctaccctcttgtgcccactttggcttaataaatcccaatccagcctagctgatttactgaagaacaaagggatgactagtttttgctaygccaaggtgagtctaggatcttaaaacttttctaagggtgccaaggagttagaatcaggggacctaagttttttctcctgaaggcatttctgagtagtag

SEQ ID NO: 58 - DNA, 201 bp, Homo sapiens
tgttgatgagaaaattactttcccttctgacattgatcctcaagttttctatgaactaccagaagcagtacaaaaggaactgctggcagagtggaagagarcaggatcagatttccacattggacataaataagcatattcagcaaaaaggtctgaaaagcaagggaataccattattttcggattagcggtttattaagc

SEQ ID NO: 59 - DNA, 201 bp, Homo sapiens
atgtgcatgtagccctttcagtaaggagtgtatggggagcttgggccctctgtggccttccatttatttgcatgtagcctcctagtcaactagggttgtamggggagcctatgtagtccctctttggttctctcatttccaaatctcctggtgacatatctggctggtccactgatctgtccttcacccgaaccacgactg

SEQ ID NO: 60 - DNA, 201 bp, Homo sapiens
gcacgtgggcctccacgattccttggcatgcgtgtcactctgggcaacaggctggtcggccacgactgcgcaggcgagaggcacagagcgactggagactktagtcccccggtttccctggagaccaggcggaagcggccggaagtagcgctgcggttggcagcggcgggatggagaagctgggggtggagccggaggagg

SEQ ID NO: 61 - DNA, 30 bp, Artificial Sequence
  primer
acgttggatgccaagtagggaatcaggatc

SEQ ID NO: 62 - DNA, 30 bp, Artificial Sequence
  Homo sapiens
acgttggatgggtctccactgatatttggg

SEQ ID NO: 63 - DNA, 24 bp, Artificial Sequence
  primer
ggatctttcatctcattattaggt

SEQ ID NO: 64 - DNA, 30 bp, Artificial Sequence
  primer
acgttggatggttagaaagcagcactgtgg

SEQ ID NO: 65 - DNA, 30 bp, Artificial Sequence
  primer
acgttggatgtcttaggaggtcattggtgc

SEQ ID NO: 66 - DNA, 17 bp, Artificial Sequence
  primer
tggcagctctcagaagg

SEQ ID NO: 67 - DNA, 31 bp, Artificial Sequence
  primer
acgttggatggtaggattctaccttagaagc

SEQ ID NO: 68 - DNA, 30 bp, Artificial Sequence
  primer
acgttggatgtaaggagtttcagtgcaccc

SEQ ID NO: 69 - DNA, 17 bp, Artificial Sequence
  primer
accttagaagcttgggg

SEQ ID NO: 70 - DNA, 30 bp, Artificial Sequence
  primer
acgttggatgttccagtcctagtgaaacgg

SEQ ID NO: 71 - DNA, 30 bp, Artificial Sequence
  primer
acgttggatgaccctggtgtctgacttttg

SEQ ID NO: 72 - DNA, 19 bp, Artificial Sequence
  primer
aacgggagagaaagaaggt

SEQ ID NO: 73 - DNA, 31 bp, Artificial Sequence
  primer
acgttggatgcacagttgtaaagactttgcc

SEQ ID NO: 74 - DNA, 30 bp, Artificial Sequence
  primer
acgttggatggtggtttgataatttccaag

SEQ ID NO: 75 - DNA, 20 bp, Artificial Sequence
  primer
tgcctaatttctggcaacta

SEQ ID NO: 76 - DNA, 31 bp, Artificial Sequence
  primer
acgttggatggatggacttaagagatcatcc

SEQ ID NO: 77 - DNA, 30 bp, Artificial Sequence
  primer
acgttggatgtgaacatcagcagctttccg

SEQ ID NO: 78 - DNA, 17 bp, Artificial Sequence
  primer
agagatcatcccaacct

SEQ ID NO: 79 - DNA, 30 bp, Artificial Sequence
  primer
acgttggatgacacttgtttgatgcctgcc

SEQ ID NO: 80 - DNA, 30 bp, Artificial Sequence
  primer
acgttggatgtttacagactgtcctctgcg

SEQ ID NO: 81 - DNA, 18 bp, Artificial Sequence
  primer
ctgcctctagggcaatga

SEQ ID NO: 82 - DNA, 30 bp, Artificial Sequence
  primer
acgttggatgagactgcgagtcacgtgcga

SEQ ID NO: 83 - DNA, 30 bp, Artificial Sequence
  primer
acgttggatggctttgttttctccacccac

SEQ ID NO: 84 - DNA, 17 bp, Artificial Sequence
  primer
gacagaggaggcggagc

SEQ ID NO: 85 - DNA, 30 bp, Artificial Sequence
  primer
acgttggatgaagcctttcttaagctctgg

SEQ ID NO: 86 - DNA, 30 bp, Artificial Sequence
  primer
acgttggatgcttcctcatctctctttccc

SEQ ID NO: 87 - DNA, 21 bp, Artificial Sequence
  primer
agctctggtaacttgcatata

SEQ ID NO: 88 - DNA, 30 bp, Artificial Sequence
  primer
acgttggatgtttcaagccaccattgcagc

SEQ ID NO: 89 - DNA, 30 bp, Artificial Sequence
  primer
acgttggatgtcaaagtctgtgtgctcatg

SEQ ID NO: 90 - DNA, 17 bp, Artificial Sequence
  primer
ggtttgtctgaggcacc

SEQ ID NO: 91 - DNA, 30 bp, Artificial Sequence
  primer
acgttggatgctcattagactcagaaggcg

SEQ ID NO: 92 - DNA, 30 bp, Artificial Sequence
  primer
acgttggatgccatctctccttcctcttac

SEQ ID NO: 93 - DNA, 19 bp, Artificial Sequence
  primer
aggtcacctgagtaagcag

SEQ ID NO: 94 - DNA, 30 bp, Artificial Sequence
  primer
acgttggatgaatggcctgttaaacctagc

SEQ ID NO: 95 - DNA, 32 bp, Artificial Sequence
  primer
acgttggatggcatatcaagaaataaaagacc

SEQ ID NO: 96 - DNA, 23 bp, Artificial Sequence
  primer
gctaaaactaagtgatttttttg

SEQ ID NO: 97 - DNA, 30 bp, Artificial Sequence
  primer
acgttggatgtttctgagggcaagaacctg

SEQ ID NO: 98 - DNA, 30 bp, Artificial Sequence
  primer
acgttggatgtggagaagtttcttaggtgg

SEQ ID NO: 99 - DNA, 21 bp, Artificial Sequence
  primer
ttacattctgaggaccataca

SEQ ID NO: 100 - DNA, 30 bp, Artificial Sequence
  primer
acgttggatgtcaggctaaattgtcggctc

SEQ ID NO: 101 - DNA, 31 bp, Artificial Sequence
  primer
acgttggatgcaaagacctgtgctaacattc

SEQ ID NO: 102 - DNA, 22 bp, Artificial Sequence
  primer
gtcggctctaagaacatgaagt

SEQ ID NO: 103 - DNA, 30 bp, Artificial Sequence
  primer
acgttggatgcagtgtacaactcttcacag

SEQ ID NO: 104 - DNA, 30 bp, Artificial Sequence
  primer
acgttggatgacagagacaaggagagaagc

SEQ ID NO: 105 - DNA, 20 bp, Artificial Sequence
  primer
cttcacagagcaatcgctca

SEQ ID NO: 106 - DNA, 29 bp, Artificial Sequence
  primer
acgttggatgctccgcggtcatggagatc

SEQ ID NO: 107 - DNA, 30 bp, Artificial Sequence
  primer
acgttggatgttacccagttctgcgcgtta

SEQ ID NO: 108 - DNA, 17 bp, Artificial Sequence
  primer
ctgtgcaagaaagtcaa

SEQ ID NO: 109 - DNA, 30 bp, Artificial Sequence
  primer
acgttggatgggcaaggaactaagaggtag

SEQ ID NO: 110 - DNA, 30 bp, Artificial Sequence
  primer
acgttggatgacctgtaacccaggagaatg

SEQ ID NO: 111 - DNA, 18 bp, Artificial Sequence
  primer
tctagacgatggaaactt

SEQ ID NO: 112 - DNA, 30 bp, Artificial Sequence
  primer
acgttggatgacagtagccagtactcaatc

SEQ ID NO: 113 - DNA, 30 bp, Artificial Sequence
  primer
acgttggatgtggccttttgccttatttcc

SEQ ID NO: 114 - DNA, 17 bp, Artificial Sequence
  primer
cagttcctaaggcccaa

SEQ ID NO: 115 - DNA, 30 bp, Artificial Sequence
  primer
acgttggatgtcaagaaagtctgggtcctc

SEQ ID NO: 116 - DNA, 31 bp, Artificial Sequence
  primer
acgttggatgctcagattaagatgtcttggg

SEQ ID NO: 117 - DNA, 17 bp, Artificial Sequence
  primer
ctgggtcctcagtacta

SEQ ID NO: 118 - DNA, 30 bp, Artificial Sequence
  primer
acgttggatgctagagaaacccatagcaac

SEQ ID NO: 119 - DNA, 30 bp, Artificial Sequence
  primer
acgttggatgcaagaggaaggacagatggc

SEQ ID NO: 120 - DNA, 17 bp, Artificial Sequence
  primer
ttgatggtcagtggtcc

SEQ ID NO: 121 - DNA, 31 bp, Artificial Sequence
  primer
acgttggatggtgggaaattgtactcattgg

SEQ ID NO: 122 - DNA, 30 bp, Artificial Sequence
  primer
acgttggatgtctggaaaatatatcgcctc

SEQ ID NO: 123 - DNA, 20 bp, Artificial Sequence
  primer
ttaaagcaaaggttttgtct

SEQ ID NO: 124 - DNA, 30 bp, Artificial Sequence
  primer
acgttggatgacaggcaccaacctctctag

SEQ ID NO: 125 - DNA, 30 bp, Artificial Sequence
  primer
acgttggatgggagcctttcaaggaacctg

SEQ ID NO: 126 - DNA, 20 bp, Artificial Sequence
  primer
tagcacaggggttcctccaa

SEQ ID NO: 127 - DNA, 30 bp, Artificial Sequence
  primer
acgttggatggcttctagacgttgtttctg

SEQ ID NO: 128 - DNA, 30 bp, Artificial Sequence
  primer
acgttggatgtgctccattttgccatcttc

SEQ ID NO: 129 - DNA, 19 bp, Artificial Sequence
  primer
tgtagatgtaaaggctgtt

SEQ ID NO: 130 - DNA, 30 bp, Artificial Sequence
  primer
acgttggatgacagcaagtccgcattcatc

SEQ ID NO: 131 - DNA, 30 bp, Artificial Sequence
  primer
acgttggatgcaaactatgctcttactagg

SEQ ID NO: 132 - DNA, 23 bp, Artificial Sequence
  primer
agcatcatctaagacataaaaag

SEQ ID NO: 133 - DNA, 30 bp, Artificial Sequence
  primer
acgttggatggaatcagaggtcctagtcag

SEQ ID NO: 134 - DNA, 30 bp, Artificial Sequence
  primer
acgttggatgttgactagattcacggctcc

SEQ ID NO: 135 - DNA, 17 bp, Artificial Sequence
  primer
tcctagtcagccttccc

SEQ ID NO: 136 - DNA, 30 bp, Artificial Sequence
  primer
acgttggatggtgaggccgctggttctaac

SEQ ID NO: 137 - DNA, 30 bp, Artificial Sequence
  primer
acgttggatgtgagcattaaaccagggcag

SEQ ID NO: 138 - DNA, 20 bp, Artificial Sequence
  primer
ccgctggttctaaccaagag

SEQ ID NO: 139 - DNA, 30 bp, Artificial Sequence
  primer
acgttggatgtctatgtgcccttctgcttc

SEQ ID NO: 140 - DNA, 28 bp, Artificial Sequence
  primer
acgttggatggctgaaacagcaagccca

SEQ ID NO: 141 - DNA, 18 bp, Artificial Sequence
  primer
ctctcacctgggccaccc

SEQ ID NO: 142 - DNA, 30 bp, Artificial Sequence
  primer
acgttggatgatctccttctgtcccttctg

SEQ ID NO: 143 - DNA, 30 bp, Artificial Sequence
  primer
acgttggatggaagccatgctgttaacagt

SEQ ID NO: 144 - DNA, 17 bp, Artificial Sequence
  primer
tgtcccttctgatggca

SEQ ID NO: 145 - DNA, 30 bp, Artificial Sequence
  primer
acgttggatgacgtaaaccagaactgaggc

SEQ ID NO: 146 - DNA, 30 bp, Artificial Sequence
  primer
acgttggatgttcttcgccttgcaatccag

SEQ ID NO: 147 - DNA, 20 bp, Artificial Sequence
  primer
tcagaggctgaggcggggag

SEQ ID NO: 148 - DNA, 30 bp, Artificial Sequence
  primer
acgttggatgtcactttaggccaactgctc

SEQ ID NO: 149 - DNA, 30 bp, Artificial Sequence
  primer
acgttggatgtttttacccagccaggtctc

SEQ ID NO: 150 - DNA, 17 bp, Artificial Sequence
  primer
agaaactggacccagtg

SEQ ID NO: 151 - DNA, 30 bp, Artificial Sequence
  primer
acgttggatgcctcatatttacgggagggc

SEQ ID NO: 152 - DNA, 30 bp, Artificial Sequence
  primer
acgttggatgaggcctgccagaattgaaac

SEQ ID NO: 153 - DNA, 17 bp, Artificial Sequence
  primer
gagggcttcaaaagtaa

SEQ ID NO: 154 - DNA, 30 bp, Artificial Sequence
  primer
acgttggatgaatgtaggatgccccagatg

SEQ ID NO: 155 - DNA, 30 bp, Artificial Sequence
  primer
acgttggatgcagctgctgtgagttagttc

SEQ ID NO: 156 - DNA, 23 bp, Artificial Sequence
  primer
ttgtcagaaatgaaaaatacatc

SEQ ID NO: 157 - DNA, 30 bp, Artificial Sequence
  primer
acgttggatgtgtttactttgccaccaacc

SEQ ID NO: 158 - DNA, 30 bp, Artificial Sequence
  primer
acgttggatgagtgccaaacctacagaagc

SEQ ID NO: 159 - DNA, 17 bp, Artificial Sequence
  primer
tgccaccaacctgccct

SEQ ID NO: 160 - DNA, 30 bp, Artificial Sequence
  primer
acgttggatgcattggcaaagtccagtcac

SEQ ID NO: 161 - DNA, 30 bp, Artificial Sequence
  primer
acgttggatgagtctaagtacccactcagc

SEQ ID NO: 162 - DNA, 17 bp, Artificial Sequence
  primer
agtcaccaaagcagttt

SEQ ID NO: 163 - DNA, 30 bp, Artificial Sequence
  primer
acgttggatgatgagtgccgggctccagaa

SEQ ID NO: 164 - DNA, 30 bp, Artificial Sequence
  primer
acgttggatgagaaggaggaacagaggcac

SEQ ID NO: 165 - DNA, 17 bp, Artificial Sequence
  primer
cttcccttcttgggatt

SEQ ID NO: 166 - DNA, 30 bp, Artificial Sequence
  primer
acgttggatgtctttgctcttccactccag

SEQ ID NO: 167 - DNA, 30 bp, Artificial Sequence
  primer
acgttggatggaaaagggacaagaggaacg

SEQ ID NO: 168 - DNA, 20 bp, Artificial Sequence
  primer
ccactccagtccttaagcga

SEQ ID NO: 169 - DNA, 30 bp, Artificial Sequence
  primer
acgttggatgtgagatctcgggaaagagtc

SEQ ID NO: 170 - DNA, 30 bp, Artificial Sequence
  primer
acgttggatgaaaccagaggtgacttttcc

SEQ ID NO: 171 - DNA, 24 bp, Artificial Sequence
  primer
aaagagtctctttataaatatggg

SEQ ID NO: 172 - DNA, 30 bp, Artificial Sequence
  primer
acgttggatgtggataaggtcacctgatgc

SEQ ID NO: 173 - DNA, 30 bp, Artificial Sequence
  primer
acgttggatgagtgtgtgcaggttgtagtc

SEQ ID NO: 174 - DNA, 17 bp, Artificial Sequence
  primer
tacacacagctccgaat

SEQ ID NO: 175 - DNA, 30 bp, Artificial Sequence
  primer
acgttggatgggtttagatggaaggaacgg

SEQ ID NO: 176 - DNA, 30 bp, Artificial Sequence
  primer
acgttggatgcagctctgtctgataaaggg

SEQ ID NO: 177 - DNA, 20 bp, Artificial Sequence
  primer
aagtggcgtgtatgtgtaat

SEQ ID NO: 178 - DNA, 30 bp, Artificial Sequence
  primer
acgttggatgccctcatgttactaacaggc

SEQ ID NO: 179 - DNA, 30 bp, Artificial Sequence
  primer
acgttggatggcagcagcctttaatgcatc

SEQ ID NO: 180 - DNA, 20 bp, Artificial Sequence
  primer
gttactaacaggctgtaact

SEQ ID NO: 181 - DNA, 30 bp, Artificial Sequence
  primer
acgttggatggtctataaagcagacttccc

SEQ ID NO: 182 - DNA, 30 bp, Artificial Sequence
  primer
acgttggatggcacaatgtctgacatctgg

SEQ ID NO: 183 - DNA, 19 bp, Artificial Sequence
  primer
agacttcccatggatgttt

SEQ ID NO: 184 - DNA, 30 bp, Artificial Sequence
  primer
acgttggatgaatgtcatcagtcaaggccc

SEQ ID NO: 185 - DNA, 30 bp, Artificial Sequence
  primer
acgttggatgtgatctcagtgacgatgcag

SEQ ID NO: 186 - DNA, 17 bp, Artificial Sequence
  primer
tgaggcctggggagacc

SEQ ID NO: 187 - DNA, 30 bp, Artificial Sequence
  primer
acgttggatgggaaagatatagccaccagg

SEQ ID NO: 188 - DNA, 30 bp, Artificial Sequence
  primer
acgttggatgaatggaagtcctccatcagc

SEQ ID NO: 189 - DNA, 18 bp, Artificial Sequence
  primer
caggagtagttatgagga

SEQ ID NO: 190 - DNA, 30 bp, Artificial Sequence
  primer
acgttggatgcacagtgagaaaaggaagag

SEQ ID NO: 191 - DNA, 30 bp, Artificial Sequence
  primer
acgttggatgtgcctgttgccaaatgcttg

SEQ ID NO: 192 - DNA, 22 bp, Artificial Sequence
  primer
aaggaagagaaagataaaatca

SEQ ID NO: 193 - DNA, 30 bp, Artificial Sequence
  primer
acgttggatggtaagcttggctctgtttcc

SEQ ID NO: 194 - DNA, 30 bp, Artificial Sequence
  primer
acgttggatgtgaaatcccaaaagcccagg

SEQ ID NO: 195 - DNA, 23 bp, Artificial Sequence
  primer
gctctgtttccaaactcataaaa

SEQ ID NO: 196 - DNA, 31 bp, Artificial Sequence
  primer
acgttggatggtcaattggcctaattttccc

SEQ ID NO: 197 - DNA, 30 bp, Artificial Sequence
  primer
acgttggatggagtggaattgttccaggtc

SEQ ID NO: 198 - DNA, 22 bp, Artificial Sequence
  primer
cattttaatttctccagagctt

SEQ ID NO: 199 - DNA, 30 bp, Artificial Sequence
  primer
acgttggatgcaaacaggaaccagaccatc

SEQ ID NO: 200 - DNA, 30 bp, Artificial Sequence
  primer
acgttggatgagcaacctcacattgacacg

SEQ ID NO: 201 - DNA, 17 bp, Artificial Sequence
  primer
accagaccatcagatcc

SEQ ID NO: 202 - DNA, 30 bp, Artificial Sequence
  primer
acgttggatgaggtttgggaacactggttg

SEQ ID NO: 203 - DNA, 30 bp, Artificial Sequence
  primer
acgttggatgggaacgtttgtcctaatggg

SEQ ID NO: 204 - DNA, 20 bp, Artificial Sequence
  primer
acaagccactgatgagggag

SEQ ID NO: 205 - DNA, 30 bp, Artificial Sequence
  primer
acgttggatgttttctggttacccaataag

SEQ ID NO: 206 - DNA, 31 bp, Artificial Sequence
  primer
acgttggatgcaatgacccaagtatctagag

SEQ ID NO: 207 - DNA, 18 bp, Artificial Sequence
  primer
atcatgtgactttccaca

SEQ ID NO: 208 - DNA, 30 bp, Artificial Sequence
  primer
acgttggatgagtgcagacactatctgctc

SEQ ID NO: 209 - DNA, 30 bp, Artificial Sequence
  primer
acgttggatgaagtgatgcagcaatgctgg

SEQ ID NO: 210 - DNA, 20 bp, Artificial Sequence
  primer
cacctcccccactagtactc

SEQ ID NO: 211 - DNA, 30 bp, Artificial Sequence
  primer
acgttggatgagagtgcctgctcagataac

SEQ ID NO: 212 - DNA, 30 bp, Artificial Sequence
  primer
acgttggatgtcaagaggtaaaaggagctg

SEQ ID NO: 213 - DNA, 17 bp, Artificial Sequence
  primer
tgctcatccctgggagt

SEQ ID NO: 214 - DNA, 30 bp, Artificial Sequence
  primer
acgttggatggtagggactttctttgctgg

SEQ ID NO: 215 - DNA, 30 bp, Artificial Sequence
  primer
acgttggatgtgtaaaaactctgggaaggg

SEQ ID NO: 216 - DNA, 20 bp, Artificial Sequence
  primer
ctcatccccaagaattattt

SEQ ID NO: 217 - DNA, 30 bp, Artificial Sequence
  primer
acgttggatgctgcggtatgagtctgtatc

SEQ ID NO: 218 - DNA, 30 bp, Artificial Sequence
  primer
acgttggatgctctgtgctccaagtacaac

SEQ ID NO: 219 - DNA, 21 bp, Artificial Sequence
  primer
aacctttcacttcccttcaaa

SEQ ID NO: 220 - DNA, 30 bp, Artificial Sequence
  primer
acgttggatgttagcccaagccatcagtgc

SEQ ID NO: 221 - DNA, 30 bp, Artificial Sequence
  primer
acgttggatgctcacccttaagcctatctc

SEQ ID NO: 222 - DNA, 17 bp, Artificial Sequence
  primer
gcatttctgtcatcgag

SEQ ID NO: 223 - DNA, 30 bp, Artificial Sequence
  primer
acgttggatgacatcctcacagtatgtccc

SEQ ID NO: 224 - DNA, 30 bp, Artificial Sequence
  primer
acgttggatgtcactgtcatgactgctcac

SEQ ID NO: 225 - DNA, 20 bp, Artificial Sequence
  primer
caagtatcttctgcccttcc

SEQ ID NO: 226 - DNA, 29 bp, Artificial Sequence
  primer
acgttggatgaacagcttcaccacggcgg

SEQ ID NO: 227 - DNA, 30 bp, Artificial Sequence
  primer
acgttggatgaaaggagtcccgagtgggag

SEQ ID NO: 228 - DNA, 20 bp, Artificial Sequence
  primer
cttcaccacggcggtcatgt

SEQ ID NO: 229 - DNA, 30 bp, Artificial Sequence
  primer
acgttggatgtaactccttggcacccttag

SEQ ID NO: 230 - DNA, 30 bp, Artificial Sequence
  primer
acgttggatgctgaagaacaaagggatgac

SEQ ID NO: 231 - DNA, 17 bp, Artificial Sequence
  primer
cctagactcaccttggc

SEQ ID NO: 232 - DNA, 31 bp, Artificial Sequence
  primer
acgttggatggcttttcagacctttttgctg

SEQ ID NO: 233 - DNA, 30 bp, Artificial Sequence
  primer
acgttggatgaaaaggaactgctggcagag

SEQ ID NO: 234 - DNA, 18 bp, Artificial Sequence
  primer
tgtggaaatctgatcctg

SEQ ID NO: 235 - DNA, 30 bp, Artificial Sequence
  primer
acgttggatgtgagagaaccaaagagggac

SEQ ID NO: 236 - DNA, 30 bp, Artificial Sequence
  primer
acgttggatgttgcatgtagcctcctagtc

SEQ ID NO: 237 - DNA, 17 bp, Artificial Sequence
  primer
gactacataggctcccc

SEQ ID NO: 238 - DNA, 30 bp, Artificial Sequence
  primer
acgttggatgatgcgtgtcactctgggcaa

SEQ ID NO: 239 - DNA, 29 bp, Artificial Sequence
  primer
acgttggatgtggtctccagggaaaccgg

SEQ ID NO: 240 - DNA, 17 bp, Artificial Sequence
  primer
cagagcgactggagact

SEQ ID NO: 241 - DNA, 200 bp, Homo sapiens
ctataaatagggcatcgtgacccggccaggggaagaagctgccgttgttctgggtactacagcagaaggtaagccgggggccccctcagctccttctcggycttgtctctctcagatgtaactgagctgtgggctaggaggaaaaggccgggaggaggcacggtgatgactgaaaaacctctcccctctcataagaccag

SEQ ID NO: 242 - DNA, 200 bp, Homo sapiens
tgagagagacaaggccgagaaggagctgagggggcccccggcttaccttctgctgtagtacccagaacaacggcagcttcttcccccggccgggtcacgakgccctatttatagctgaggggtggggatggagctgttcccaggctgcctgtgcacaggctggagaggagggttacatcacttggccagaccacaggctg

SEQ ID NO: 243 - DNA, 200 bp, Homo sapiens
tgtagtgtctgtaataccttagttttttccacagatgcttgtgatttttgaacaatacgtgaaagatgcaagcacctgaatttctgtttgaatgcggaacmatagctggttatttctcccttgtgttagtaataaacgtcttgccacaataagcctccaaaaattttatctttcatttagcagccaaacagatgtataca

SEQ ID NO: 244 - DNA, 200 bp, Homo sapiens
ctcccacgctctctggacttcacagaactggatgttgctgctgagaagattgacaggttcatgcaggctgtgacaggatggaagactggctgctccctgaygggagccagtgtggacagcaccctggctttcaacacctacgtccacttccaaggtaaggcaaacctctctgctggctctggccctaggacttagtatcc

SEQ ID NO: 245 - DNA, 30 bp, Artificial Sequence
  primer
acgttggatggcccacagctcagttacatc

SEQ ID NO: 246 - DNA, 30 bp, Artificial Sequence
  primer
acgttggatgactacagcagaaggtaagcc

SEQ ID NO: 247 - DNA, 21 bp, Artificial Sequence
  primer
gttacatctgagagagacaag

SEQ ID NO: 248 - DNA, 30 bp, Artificial Sequence
  primer
acgttggatgtgtagtacccagaacaacgg

SEQ ID NO: 249 - DNA, 30 bp, Artificial Sequence
  primer
acgttggatgagcctgggaacagctccatc

SEQ ID NO: 250 - DNA, 20 bp, Artificial Sequence
  primer
ttcccccggccgggtcacga

SEQ ID NO: 251 - DNA, 30 bp, Artificial Sequence
  primer
acgttggatgaggcttattgtggcaagacg

SEQ ID NO: 252 - DNA, 30 bp, Artificial Sequence
  primer
acgttggatggtgaaagatgcaagcacctg

SEQ ID NO: 253 - DNA, 20 bp, Artificial Sequence
  Homo sapiens
agggagaaataaccagctat

SEQ ID NO: 254 - DNA, 30 bp, Artificial Sequence
  primer
acgttggatggattgacaggttcatgcagg

SEQ ID NO: 255 - DNA, 30 bp, Artificial Sequence
  primer
acgttggatgacgtaggtgttgaaagccag

SEQ ID NO: 256 - DNA, 21 bp, Artificial Sequence
  primer
ggaagactggctgctccctga

(19) United States
(12) Patent Application Publication
(10) Pub. No.: US 2006/0257913 A1
(43) Pub. Date: Nov. 16, 2006

(54) Genetic polymorphisms associated with myocardial infarction and uses thereof

(75) Inventors: Byung-chul Kim, Hwaseong-si (KR); Yeon-Su Lee, Goyang-si (KR); Min-sun Kim, Hwaseong-si (KR); Ok-kyoung Son, Seoul (KR); Moon-sue Lee, Seoul (KR); Ki-eun Kim, Seoul (KR); Ok-ryul Song, Seoul (KR); Hyo-jeong Jeon, Seoul (KR); Kyung-Hee Park, Seoul (KR); Tae-jin Ahn, Seoul (KR); Jae-Heup Kim, Seoul (KR)

Correspondence Address: BUCHANAN, INGERSOLL & ROONEY PC, POST OFFICE BOX 1404, ALEXANDRIA, VA, 22313-1404, US

(73) Assignee: SAMSUNG ELECTRONICS CO., LTD., Suwon-si (KR)

(21) Appl. No.: 11/430,939

(22) Filed: May 10, 2006

(30) Foreign Application Priority Data
May 13, 2005 (KR) 10-2005-0040163
Jun. 2, 2005 (KR) 10-2005-0047195

(51) Int. Cl. C12Q 1/68; G01N 33/53; C07H 21/04; C12P 21/06; C07K 14/47

(52) U.S. Cl. 435/6; 435/7.1; 435/69.1; 435/320.1; 435/325; 530/350; 536/23.5

(57) ABSTRACT

A genetic polymorphism associated with myocardial infarction is provided. More particularly, provided are a polynucleotide including a single nucleotide polymorphism (SNP) associated with myocardial infarction, a polynucleotide hybridized with the polynucleotide, a polypeptide encoded by one of the polynucleotides, an antibody bound to the polypeptide, a microarray and a kit including one of the polynucleotides, a myocardial infarction diagnosis method, a SNP detecting method and a method of screening pharmaceutical compositions for myocardial infarction.

### CROSS-REFERENCE TO RELATED PATENT APPLICATION

This application claims the benefit of Korean Patent Application Nos. 10-2005-0040163 filed on May 13, 2005 and 10-2005-0047195 filed on Jun. 2, 2005, in the Korean Intellectual Property Office, the disclosure of which is incorporated herein by reference in its entirety.

### BACKGROUND

1. Field of the Disclosure

The present disclosure relates to genetic polymorphisms associated with myocardial infarction and uses thereof.

2. Description of the Related Art

99.9% of base sequences of the human genome are identical. Diversity in individuals' appearance, behavior and susceptibility to certain diseases is caused by partial differences in the remaining 0.1% of the base sequences in the human genome. That is, differences in about 3 million base sequences of the human genome account for the diversity among individuals, communities, races and peoples. The differences in base sequences contribute to the differences in disease distributions as well as phenotypic distinctions such as skin color of different races. There are about 3 billion base pairs, with polymorphisms occurring at intervals of about 1.0 kb. That is, there are about 3 million base pairs in total that vary from person to person, and variations in these base pairs are referred to as Single Nucleotide Polymorphisms (SNPs). Causes of the diversity among individuals and communities or the difference between a disease group and a normal group may be found through the analysis of the 3 million SNPs; analysis of every base sequence is unnecessary.

The prime object of genetics is to map phenotypic differences such as diseases in humans to variations in DNA. A polymorphic marker existing in all genomes is the best means of obtaining this object. Microsatellite markers have been commonly used as polymorphic markers to distinguish individuals and find genes related to genetic diseases, but SNPs have drawn attention with the development of DNA chips. Automatic SNP detection on a large scale is possible because of the high frequency of SNPs, the safety of using SNPs, and the even distribution of SNPs across all genomes. SNPs will contribute to predictive medical science, which is a new branch of medical science. For example, a revolutionary process of predicting diseases of individuals and investigating individual's reactions to a certain medical supply can be performed by implementing up-to-date biotechnology such as a DNA chip technique and a high speed DNA sequence analysis technique.

The study of SNPs involves an analysis of genotypes evenly distributed throughout the population. By studying SNPs, a population may be divided according to genotype, and if a disease group is significantly distributed according to a genotype, the relationship between the genotype and the disease can be established. In most studies of SNPs, if a single genotype or several genotypes have significantly different distributions in a disease group and a normal group, the differences in disease frequency according to the genotype may be analyzed.

About 510,000 SNPs exist in genes, approximately one sixth of the 3 million SNPs in human genomes. It is useful to know the distribution of such SNPs in genes because these SNPs can be directly related to gene expression or protein functions. If a genotype associated with a certain disease can effect a change in a gene expression or a protein function, the gene or the protein is likely to be a cause of the disease. In this case, the gene can be the target gene for disease detection and treatment. The susceptibility to the disease may also be analyzed using SNP analysis of the gene.

SNPs in transcription regulatory regions of a gene sequence, such as a promoter, can regulate the quantity of expressed genes. On rare occasions, SNPs also influence the stability and translation efficiency of RNA located in sequences at exon-intron boundaries affecting RNA splicing or in a 3′-untranslated region (3′-UTR). Although some SNPs are located in an encoding region or a transcription and translation regulatory region, and do not affect proteins directly, such an SNP associated with a disease may be found. That is, the SNPs that are located in an encoding region or a transcription and translation regulatory region may be a useful index for determining susceptibility to a disease. SNPs which have not been found yet could be in a base sequence directly affecting gene expression or proteins, or related to and inherited with the base sequence.

Cardiovascular disease is a major cause of death in industrialized countries around the world, and has been a major cause of death in the Republic of Korea since the 1970s. According to the Korean National Statistical Office, in 2003, 22,000 out of 246,000 deaths (9087 per 100,000, or 9.1%) were the result of cardiac disorders and hyperpiesia, which are the third leading cause of death in Korea following cancer and cerebrovascular disease.

Coronary artery disease, which ranks high among cardiovascular diseases, is usually caused by arteriosclerosis, the blocking or narrowing of coronary arteries supplying blood to the heart. Blocking of the coronary artery indicates myocardial infarction and narrowing of the coronary artery indicates angina pectoris. The causes of coronary artery disease are known to be hyperlipidemia (hypercholesterolemia), hyperpiesia, smoking, diabetes, genetic inheritance, obesity, lack of exercise, stress and menopause. A subject having complex factors of coronary artery disease has a higher risk of incidence.

Currently, X-ray and ultrasonography of the interior of the heart and coronary artery can be used for cardiovascular disease diagnosis. However, as with the diagnosis or prognosis of other various cardiovascular and complicated diseases including myocardial infarction using other physical techniques, the diagnosis or prediction can be performed only when the diseases are at an advanced stage.

### SUMMARY

The present inventors have found SNPs associated with myocardial infarction, which makes it possible to predict the incidence probability of and genetic susceptibility to myocardial infarction.

The present disclosure describes single nucleotide polymorphisms (SNP) associated with myocardial infarction and provides polynucleotides comprising the SNPs. The present disclosure also provides a polynucleotide capable of specifically hybridizing with a polynucleotide containing such a SNP.

The present disclosure also provides a polypeptide encoded by the polynucleotide, and provides an antibody capable of specifically binding to the polypeptide.

The present disclosure also provides a microarray for detecting polynucleotides that comprise the SNPs, and a kit for detecting polynucleotides including the SNPs. In preferred embodiments, the micro array or kit comprises a polynucleotide that includes such a SNP, a polypeptide encoded by the polynucleotide, or an antibody capable of specifically binding to the polypeptide.

The present disclosure also provides a method of identifying a subject having an increased or decreased risk of incidence of myocardial infarction.

The present disclosure also provides a method of detecting SNPs in nucleic acid molecules.

The present disclosure also provides a method of screening pharmaceutical compositions for effects on myocardial infarction.

The present disclosure also provides a method of regulating gene expression.

According to a preferred aspect of the present disclosure, there is provided a polynucleotide comprising at least 8 contiguous nucleotides including the 101^{st} base of the nucleotide sequence of nucleotide sequences of SEQ ID NOS: 1 to 60 and 241 to 244, and complementary polynucleotides of the nucleotide sequences.

According to another aspect of the present disclosure, there is provided a polynucleotide capable of specifically hybridizing with the polynucleotide or the complementary polynucleotide thereof.

According to another aspect of the present disclosure, there is provided a polypeptide encoded by the polynucleotide, and an antibody specifically bound to the polypeptide.

According to another aspect of the present disclosure, there is provided a microarray for detecting a polynucleotide comprising the SNP, a polypeptide encoded by the polynucleotide or a cDNA of the polynucleotide. According to another aspect of the present disclosure, there is provided a kit for detecting SNPs including the polynucleotide, the polypeptide encoded by the polynucleotide or cDNA of the polynucleotide. In preferred embodiments, the micro array or kit comprises a polynucleotide that includes such a SNP, a polypeptide encoded by the polynucleotide, or an antibody capable of specifically binding to the polypeptide.

According to another aspect of the present disclosure, there is provided a method of identifying a subject having an increased or decreased risk of incidence of myocardial infarction including isolating a nucleic acid sample from the subject and determining the presence or absence of an allele at a polymorphic site of one or more polynucleotides among SEQ ID NOS: 1 to 60 and 241 to 244, wherein the polymorphic site is positioned at the 101^{st} nucleotide of the polynucleotides.

According to another aspect of the present disclosure, there is provided a method of detecting SNPs in nucleic acid molecules including contacting a test sample having nucleic acid molecules with a reagent capable of specifically hybridizing under strict conditions with a polynucleotide of nucleotide sequences of SEQ ID NOS: 1 to 60 and 241 to 244 containing at least 8 contiguous nucleotides and the 101^{st} base of the nucleotide sequence and complementary polynucleotides of the nucleotide sequences and detecting the formation of a hybridized double-strand.

According to another aspect of the present disclosure, there is provided a method of screening pharmaceutical compositions for myocardial infarction including contacting a candidate material with a polypeptide encoded by a polynucleotide of nucleotide sequences of SEQ ID NOS: 1 to 60 and 241 to 244 containing at least 8 contiguous nucleotides and the 101^{st} base of nucleotide sequence and complementary polynucleotides of the nucleotide sequences under proper conditions for the formation of a binding complex and detecting the formation of the binding complex between the polypeptide and the candidate material.

According to another aspect of the present disclosure, there is provided a method of regulating gene expression including binding an anti-sense nucleotide or Si RNA with a polynucleotide of nucleotide sequences of SEQ ID NOS: 1 to 60 and 241 to 244 containing at least 8 contiguous nucleotides and the 101^{st} base of the nucleotide sequence, wherein the anti-sense nucleotide and Si RNA is specific to the polynucleotide.

The above aspects and advantages of the present disclosure will become more apparent by describing in detail exemplary embodiments thereof.

### DETAILED DESCRIPTION

A polynucleotide containing a single nucleotide polymorphism (SNP) associated with myocardial infarction according to an aspect of the present disclosure includes a polynucleotide containing at least 8 contiguous nucleotides that include the 101^{st} base of the nucleotide sequence of SEQ ID NOS: 1 to 60 and 241 to 244, and complementary polynucleotides of the nucleotide sequences.

A polynucleotide containing one of SEQ ID NOS: 1 to 60 and 241 to 244 is a polymorphic sequence. A polymorphic sequence is a nucleotide sequence where SNPs exist, that is a nucleotide sequence containing one or more polymorphic sites in a polynucleotide sequence. The polynucleotide may be DNA or RNA. Herein, the term SNP can refer to the most commonly found single base-pair variation among DNA sequence polymorphisms shown in every 1 kb in the DNA of individuals or rarer variations.

As shown in the Examples of the present disclosure, a series of selections were made in order to find SNPs associated with cardiovascular disease, more preferably SNPs associated with myocardial infarction. DNA was isolated from blood of myocardial infarction patients and normal persons and amplified. After an analysis of the SNP sequence in the DNA, SNPs having significantly different appearance frequencies between the patients and normal persons were identified. 64 SNPs and the genotypes thereof which were identified in Examples are disclosed in Table 1 to 3.

TABLE 1

| SEQ ID | | | | | | | | | | | | GTX | |
|---|---|---|---|---|---|---|---|---|---|---|---|---|---|
| NO: | alias_id | cas_num | con_num | allele A | allele a | Cas_AA | cas_Aa | cas_aa | conga | con_Aa | con_aa | p-val | allele_OR |
| 1 | MI_0042 | 213 | 184 | A | G | 2 | 44 | 167 | 10 | 49 | 125 | 0.00777 | 0.550 |
| 2 | MI_0050 | 220 | 190 | T | G | 8 | 57 | 155 | 2 | 35 | 153 | 0.0353 | 1.74 |
| 3 | MI_0056 | 218 | 185 | T | G | 7 | 58 | 153 | 1 | 31 | 153 | 0.00669 | 2.02 |
| 4 | MI_0070 | 220 | 187 | A | G | 10 | 54 | 156 | 9 | 68 | 110 | 0.0301 | 0.677 |
| 5 | MI_0100 | 216 | 189 | C | T | 159 | 54 | 3 | 124 | 55 | 10 | 0.0397 | 1.53 |
| 6 | MI_0127 | 217 | 190 | C | T | 127 | 79 | 11 | 126 | 62 | 2 | 0.0347 | 0.693 |
| 7 | MI_0159 | 221 | 191 | A | G | 2 | 39 | 180 | 2 | 17 | 172 | 0.0221 | 1.85 |
| 8 | MI_0177 | 218 | 190 | A | G | 0 | 12 | 206 | 0 | 22 | 168 | 0.0312 | 0.461 |
| 9 | MI_0232 | 215 | 189 | C | T | 43 | 109 | 63 | 53 | 98 | 38 | 0.0450 | 0.708 |
| 10 | MI_0235 | 216 | 189 | A | G | 5 | 63 | 148 | 2 | 33 | 154 | 0.00865 | 1.87 |
| 11 | MI_0292 | 212 | 189 | T | G | 16 | 102 | 94 | 5 | 64 | 120 | 0.000255 | 1.90 |
| 12 | MI_0294 | 221 | 191 | A | G | 138 | 77 | 6 | 100 | 76 | 15 | 0.02 | 1.52 |
| 13 | MI_0299 | 220 | 191 | A | G | 36 | 110 | 74 | 61 | 85 | 45 | 0.000629 | 0.596 |
| 14 | MI_0354 | 222 | 190 | A | G | 98 | 101 | 23 | 62 | 96 | 32 | 0.027 | 1.47 |
| 15 | MI_0370 | 222 | 191 | A | G | 44 | 119 | 59 | 73 | 83 | 35 | 0.000155 | 0.584 |
| 16 | MI_0374 | 219 | 187 | A | G | 5 | 48 | 166 | 1 | 25 | 161 | 0.023 | 1.96 |
| 17 | MI_0393 | 222 | 189 | C | T | 50 | 125 | 47 | 31 | 82 | 76 | 0.000151 | 1.67 |
| 18 | MI_0433 | 222 | 191 | A | G | 61 | 106 | 55 | 72 | 86 | 33 | 0.0444 | 0.698 |
| 19 | MI_0464 | 222 | 189 | A | G | 27 | 95 | 100 | 31 | 96 | 62 | 0.0363 | 0.703 |
| 20 | MI_0493 | 221 | 191 | A | G | 85 | 102 | 34 | 61 | 82 | 48 | 0.0422 | 1.40 |

| | | | con_HWX_p- | | | | |
|---|---|---|---|---|---|---|---|
| SEQ ID NO: | allele_OR_LB | allele_OR_UB | val | gene_name | SNP_function | AA_change | AA_position |
| 1 | 0.369 | 0.82 | 0.0918 | intergenic | intergenic | n/a | n/a |
| 2 | 1.15 | 2.64 | 1.00 | LOC144678 | Intron | null | null |
| 3 | 1.30 | 3.13 | 0.641 | LOC144678 | mrna-utr | null | null |
| 4 | 0.479 | 0.958 | 0.810 | FLJ11117 | mrna-utr | null | null |
| 5 | 1.06 | 2.23 | 0.249 | intergenic | intergenic | n/a | n/a |
| 6 | 0.49 | 0.98 | 0.0402 | intergenic | intergenic | n/a | n/a |
| 7 | 1.08 | 3.18 | 0.0988 | intergenic | intergenic | n/a | n/a |
| 8 | 0.225 | 0.944 | 0.476 | intergenic | intergenic | n/a | n/a |
| 9 | 0.536 | 0.934 | 0.665 | DUSP10 | locus-region | null | null |
| 10 | 1.23 | 2.86 | 0.392 | KIAA1573 | mrna-utr | null | null |
| 11 | 1.37 | 2.63 | 0.241 | DSCR1 | Intron | null | null |
| 12 | 1.10 | 2.10 | 1 | intergenic | intergenic | n/a | n/a |
| 13 | 0.452 | 0.786 | 0.144 | intergenic | intergenic | n/a | n/a |
| 14 | 1.11 | 1.95 | 0.662 | KIAA1363 | Intron | null | null |
| 15 | 0.442 | 0.77 | 0.176 | MANBA | mrna-utr | null | null |
| 16 | 1.21 | 3.17 | 0.984 | PAPSS1 | coding-synon | K | 12 |
| 17 | 1.26 | 2.21 | 0.281 | MANBA | Intron | null | null |
| 18 | 0.529 | 0.92 | 0.45 | intergenic | intergenic | n/a | n/a |
| 19 | 0.529 | 0.934 | 0.656 | intergenic | intergenic | n/a | n/a |
| 20 | 1.06 | 1.84 | 0.0588 | FLJ40288 | mrna-utr | null | Null |

| SEQ ID | | | | | | | | | | | | GTX | |
|---|---|---|---|---|---|---|---|---|---|---|---|---|---|
| NO: | alias_id | cas_num | con_num | allele A | allele a | cas_AA | cas_Aa | cas_aa | conga | con_Aa | con_aa | p-val | allele_OR |
| 21 | MI_0495 | 221 | 191 | A | G | 136 | 68 | 17 | 90 | 84 | 17 | 0.011 | 1.49 |
| 22 | MI_0507 | 222 | 191 | T | G | 8 | 60 | 154 | 8 | 72 | 111 | 0.0498 | 0.69 |
| 23 | MI_0526 | 221 | 191 | C | A | 23 | 97 | 101 | 33 | 91 | 67 | 0.0375 | 0.685 |
| 24 | MI_0577 | 215 | 185 | A | G | 139 | 64 | 12 | 134 | 50 | 1 | 0.00782 | 0.636 |
| 25 | MI_0606 | 222 | 190 | C | G | 142 | 68 | 12 | 136 | 53 | 1 | 0.00809 | 0.647 |
| 26 | MI_0720 | 221 | 190 | A | G | 12 | 73 | 136 | 21 | 73 | 96 | 0.0299 | 0.648 |
| 27 | MI_1005 | 221 | 189 | C | A | 16 | 66 | 139 | 12 | 92 | 85 | 0.000418 | 0.643 |
| 28 | MI_1022 | 221 | 190 | A | G | 5 | 62 | 154 | 2 | 35 | 153 | 0.0397 | 1.7 |
| 29 | MI_1028 | 220 | 190 | T | G | 193 | 27 | 0 | 181 | 9 | 0 | 0.00821 | 0.371 |
| 30 | MI_1029 | 221 | 190 | C | T | 46 | 124 | 51 | 60 | 92 | 38 | 0.0468 | 0.757 |
| 31 | MI_1036 | 219 | 191 | C | T | 14 | 102 | 103 | 29 | 90 | 72 | 0.00832 | 0.667 |
| 32 | MI_1039 | 217 | 189 | A | G | 154 | 59 | 4 | 159 | 27 | 3 | 0.00415 | 0.524 |
| 33 | MI_1051 | 222 | 191 | A | G | 131 | 85 | 6 | 95 | 80 | 16 | 0.0172 | 1.48 |
| 34 | MI_1065 | 219 | 189 | A | G | 1 | 36 | 182 | 1 | 50 | 138 | 0.0193 | 0.596 |
| 35 | MI_1070 | 216 | 185 | T | G | 28 | 107 | 81 | 41 | 93 | 51 | 0.0189 | 0.675 |
| 36 | MI_1071 | 222 | 190 | T | G | 2 | 39 | 181 | 4 | 51 | 135 | 0.0384 | 0.583 |
| 37 | MI_1076 | 215 | 190 | T | G | 97 | 96 | 22 | 108 | 72 | 10 | 0.0303 | 0.662 |
| 38 | MI_1096 | 221 | 191 | A | G | 0 | 6 | 215 | 0 | 15 | 176 | 0.0235 | 0.337 |
| 39 | MI_1112 | 222 | 191 | A | G | 115 | 94 | 13 | 123 | 62 | 6 | 0.0283 | 0.649 |
| 40 | MI_1130 | 222 | 190 | C | G | 155 | 64 | 3 | 150 | 40 | 0 | 0.0359 | 0.629 |

| | | | con_HWX_p- | | | | |
|---|---|---|---|---|---|---|---|
| SEQ ID NO: | allele_OR_LB | allele_OR_UB | val | gene_name | SNP_function | AA_change | AA_position |
| 21 | 1.09 | 2.03 | 0.741 | intergenic | intergenic | n/a | n/a |
| 22 | 0.49 | 0.972 | 0.537 | GNA12 | Intron | null | null |
| 23 | 0.515 | 0.911 | 0.775 | intergenic | intergenic | n/a | n/a |
| 24 | 0.437 | 0.925 | 0.135 | ALOX5AP | Intron | null | null |
| 25 | 0.449 | 0.934 | 0.0795 | ALOX5AP | Intron | null | null |
| 26 | 0.473 | 0.887 | 0.165 | LGALS2 | Intron | null | null |
| 27 | 0.470 | 0.88 | 0.0571 | intergenic | intergenic | n/a | n/a |
| 28 | 1.12 | 2.58 | 1 | ANK3 | mrna-utr | null | null |
| 29 | 0.172 | 0.799 | 1 | HIP1 | Intron | null | null |
| 30 | 0.575 | 0.997 | 0.776 | intergenic | intergenic | n/a | n/a |
| 31 | 0.499 | 0.892 | 0.883 | intergenic | intergenic | n/a | n/a |
| 32 | 0.337 | 0.815 | 0.15 | intergenic | intergenic | n/a | n/a |
| 33 | 1.08 | 2.03 | 0.865 | intergenic | intergenic | n/a | n/a |
| 34 | 0.382 | 0.928 | 0.212 | intergenic | intergenic | n/a | n/a |
| 35 | 0.509 | 0.895 | 1 | THH | Intron | null | null |
| 36 | 0.384 | 0.888 | 1 | MAP2K4 | Intron | null | null |
| 37 | 0.486 | 0.902 | 0.692 | intergenic | intergenic | n/a | n/a |
| 38 | 0.129 | 0.877 | 1 | intergenic | intergenic | n/a | n/a |
| 39 | 0.467 | 0.901 | 0.815 | RGS7 | Intron | null | null |
| 40 | 0.415 | 0.952 | 0.23 | RBL2 | mrna-utr | null | null |

| SEQ ID | | | | | | | | | | | | GTX | |
|---|---|---|---|---|---|---|---|---|---|---|---|---|---|
| NO: | alias_id | cas_num | con_num | allele A | allele a | cas_AA | cas_Aa | cas_aa | con_AA | con_Aa | con_aa | p-val | allele_OR |
| 41 | MI_1145 | 221 | 191 | A | G | 23 | 99 | 99 | 34 | 93 | 64 | 0.0217 | 0.67 |
| 42 | MI_1169 | 212 | 186 | A | G | 3 | 37 | 172 | 0 | 19 | 167 | 0.0207 | 2.10 |
| 43 | MI_1175 | 222 | 187 | A | G | 0 | 26 | 196 | 1 | 36 | 150 | 0.0315 | 0.55 |
| 44 | MI_1186 | 219 | 188 | A | G | 149 | 64 | 6 | 97 | 73 | 18 | 0.000435 | 1.94 |
| 45 | MI_1206 | 222 | 190 | A | G | 80 | 113 | 29 | 57 | 90 | 43 | 0.035 | 1.38 |
| 46 | MI_1209 | 218 | 190 | T | G | 16 | 77 | 125 | 16 | 89 | 85 | 0.0374 | 0.713 |
| 47 | MI_1221 | 219 | 190 | A | G | 2 | 40 | 177 | 0 | 18 | 172 | 0.00955 | 2.25 |
| 48 | MI_1247 | 215 | 186 | C | T | 54 | 101 | 60 | 67 | 85 | 34 | 0.0193 | 0.661 |
| 49 | MI_1261 | 222 | 189 | C | T | 171 | 50 | 1 | 165 | 22 | 2 | 0.00513 | 0.557 |
| 50 | MI_1264 | 222 | 190 | C | T | 14 | 55 | 153 | 2 | 46 | 142 | 0.0182 | 1.52 |
| 51 | MI_1272 | 221 | 188 | A | G | 26 | 106 | 89 | 38 | 91 | 59 | 0.0332 | 0.696 |
| 52 | MI_1273 | 221 | 188 | C | A | 188 | 30 | 3 | 136 | 45 | 7 | 0.00547 | 2.10 |
| 53 | MI_1329 | 220 | 189 | C | G | 71 | 115 | 34 | 93 | 74 | 22 | 0.00255 | 0.637 |
| 54 | MI_1363 | 221 | 190 | T | A | 103 | 102 | 16 | 67 | 97 | 26 | 0.0199 | 1.48 |
| 55 | MI_1377 | 222 | 190 | C | T | 86 | 108 | 28 | 100 | 72 | 18 | 0.0181 | 0.678 |
| 56 | MI_1503 | 216 | 187 | A | G | 0 | 30 | 186 | 2 | 42 | 143 | 0.0153 | 0.532 |

| | | | con_HWX_p- | | | | |
|---|---|---|---|---|---|---|---|
| SEQ ID NO: | allele_OR_LB | allele_OR_UB | val | gene_name | SNP_function | AA_change | AA_position |
| 41 | 0.504 | 0.89 | 1 | intergenic | intergenic | n/a | n/a |
| 42 | 1.20 | 3.67 | 1 | SIPA1L1 | Intron | null | null |
| 43 | 0.327 | 0.924 | 0.413 | intergenic | intergenic | n/a | n/a |
| 44 | 1.39 | 2.71 | 0.38 | intergenic | intergenic | n/a | n/a |
| 45 | 1.04 | 1.82 | 0.476 | CSMD1 | Intron | null | null |
| 46 | 0.525 | 0.969 | 0.316 | intergenic | intergenic | n/a | n/a |
| 47 | 1.27 | 3.96 | 1 | LOC387895 | Intron | null | null |
| 48 | 0.499 | 0.874 | 0.449 | intergenic | intergenic | n/a | n/a |
| 49 | 0.34 | 0.911 | 0.212 | intergenic | intergenic | n/a | n/a |
| 50 | 1.04 | 2.22 | 0.746 | MST1R | coding-nonsynon | G | 1335 |
| 51 | 0.525 | 0.923 | 0.769 | SLC8A1 | Intron | null | null |
| 52 | 1.35 | 3.26 | 0.0919 | intergenic | intergenic | n/a | n/a |
| 53 | 0.478 | 0.851 | 0.168 | NFKB1 | Intron | null | null |
| 54 | 1.11 | 1.98 | 0.361 | intergenic | intergenic | n/a | n/a |
| 55 | 0.505 | 0.91 | 0.369 | NFKB1 | Intron | null | null |
| 56 | 0.328 | 0.862 | 1 | CYBA | coding-nonsynon | H | 72 |

In Table 1, ‘SEQ ID NO:’ is the sequence identification number of a polynucleotide including the SNP in the sequence listing of the present disclosure.

‘Alias_id’ is a SNP number arbitrarily designated by the inventors of the present disclosure.

‘Cas_num’ and ‘con_num’ respectively indicate the number of patients and normal persons having the SNP.

Allele ‘A’ and ‘a’ respectively represent a low mass allele and a high mass allele in sequencing experiments according to a homogeneous MassEXTEND™ technique of Sequenom, and are arbitrarily designated for convenience of experiments.

‘Cas AA’, ‘cas_Aa’ and ‘cas_aa’ respectively represent the number of patients having the genotype ‘AA’, ‘Aa’ and ‘aa’. Also, ‘con_AA’, ‘con_Aa’ and ‘con_aa’ respectively represent the number of normal persons having the genotype ‘AA’, ‘Aa’ and ‘aa’.

‘GTX_p-val’ is the p-value obtained by inspecting the gene using Fisher's exact test. When the p-value was 0.05 or less, it was determined that the genotype between the disease group and the normal group was not identical, i.e. significant.

‘Allele_OR’ is the odds ratio of the probability of the SNP in the disease group to the probability of the SNP in the normal group based on genotype.

‘Allele_OR_LB’ and ‘allele_OR_UB’ respectively indicate the lower limit and the upper limit of the 95% confidence interval of the odds ratio. When the odds ratio exceeds 1, ‘A’ is the risk factor and when the odds ratio is less than 1, ‘a’ is the risk factor. When the confidence interval includes 1, it cannot be determined that the relationship between the genotype and the disease is significant.

‘Con_HWX_p-val’ indicates the Hydy-Weinberg Equilibrium in the normal group. When the p-value was 0.05 or less, the normal group was not determined to be in Hardy-Weinberg Equilibrium.

‘Gene_name’ is the name of the gene to which the SNP belongs.

‘SNP_function’ is the role performed by the SNP within the gene.

‘A_change’ indicates whether an amino acid is changed by the SNP.

‘AA_position’ indicates a position in the polypeptide of an amino acid coded by the SNP site.

TABLE 2

| SEQ ID | | | | | | | | |
|---|---|---|---|---|---|---|---|---|
| NO: | alias_id | allele A | allele a | gene_name | SNP_function | AA_change | AA_position | cas_a |
| 57 | MI_1111 | A | G | polymerase iota | intron | null | null | 0.287 |
| 58 | MI_1248 | C | T | polymerase iota | exon | Thr->Ala | 706 | 0.715 |
| 59 | MI_2143 | T | G | polymerase iota | intron | null | null | 0.285 |
| 60 | MI_2144 | T | G | polymerase iota | intron | null | null | 0.322 |

| SEQ ID | | | | | | | | |
|---|---|---|---|---|---|---|---|---|
| NO: | con_a | Delta | chi_value | chi_exact_Pvalue | OR | OR_LB | OR_UB | con_HW |
| 57 | 0.215 | 0.072 | 7.561 | 0.0228 | 1.47 | 1.113 | 1.937 | HWE |
| 58 | 0.788 | 0.075 | 8.043 | 0.0179 | 1.5 | 1.131 | 1.978 | HWE |
| 59 | 0.217 | 0.068 | 6.829 | 0.0329 | 1.49 | 1.094 | 1.904 | HWE |
| 60 | 0.242 | 0.8 | 8.816 | 0.0122 | 1.49 | 1.137 | 1.949 | HWE |

TABLE 3

| SEQ ID | GenBank accession | | | | | | Chi_exact_p- |
|---|---|---|---|---|---|---|---|
| NO: | No. of SNP in NCBI | allele A | allele a | OR | OR_LB | OR_UB | Value |
| 241 | rs2148582 | A | G | 0.619 | 0.436 | 0.877 | 0.0296 |
| 242 | rs5050 | T | G | 0.659 | 0.455 | 0.955 | 0.0281 |
| 243 | rs7079 | T | G | 0.657 | 0.428 | 1.01 | 0.0583 |
| 244 | rs699 | G | A | 1.61 | 1.14 | 2.29 | 0.0297 |

‘Cas_a’, ‘con_a’ and ‘Delta’ respectively indicate the frequency of ‘a’ in the disease group, the frequency of ‘a’ in the normal group, and the absolute value of the difference between ‘cas_a’ and ‘con_a’. Herein, ‘cas_a’ is given by (the frequency of the genotype ‘aa’×2+the frequency of the genotype ‘Aa’)/(the number of samples of the disease group×2) and ‘con_a’ is given by (the frequency of the genotype ‘aa’×2+the frequency of the genotype ‘Aa’)/(the number of samples of the normal group×2).

‘Chi-value’ is obtained through a chi-square test, and was used for p-value calculation. ‘Chi-exact-p-value’ indicates the p-value of Fisher's exact test of chi-squeare test, and is a variable used to determine statistical significance more accurately, since the chi-square test result may be inaccurate when the number of genotypes is less than 5. When the p-value was 0.05 or less, it was determined that the genotype between the disease group and the normal group was not identical, i.e. significant.

‘OR’ is a ratio noticing how often a specific genotype is found in a group of the disease group and the normal group and is calculated as (the number of patients having a specific genotype in the disease group)×(the number of persons not having the specific genotype in the normal group)/(the number of patients not having the specific genotype in the disease group)ₓ(the number of persons having the specific genotype in the normal group). ‘OR_LB’, ‘OR_UB’ respectively indicate the minimum value and the maximum value of the confidence interval of OR at a 5% level of significance.

A haplotype for diagnosis of myocardial infarction according to an aspect of the present disclosure may be composed of polynucleotides of SEQ ID NOS: 57 to 60. The SNPs' linkage disequlibriums (LD) with each other are disclosed in Table 4. As illustrated in Table 4, the four SNPs composed a strong LD block.

TABLE 4

| | MI_2144 | MI_1111 | MI_2143 | MI_1248 |
|---|---|---|---|---|
| MI_2144 | 0 | 1 | 1 | 0.9898 |
| MI_1111 | 1 | 0 | 1 | 0.9951 |
| MI_2143 | 1 | 1 | 0 | 1 |
| MI_1248 | 0.9898 | 0.9951 | 1 | 0 |

For example, the 101^{st} base, which is the SNP site, of the polynucleotides of SEQ ID NOS: 57 to 60 composing the haplotype can be the risk allele. That is, the haplotype can be a haplotype No. 1 or 2 in Table 5.

TABLE 5

| Haplotype | | | | | | | Hap.Freq |
|---|---|---|---|---|---|---|---|
| No. | MI_2144 | MI_1111 | MI_2143 | MI_1248 | Hap.score | p.val | total_freq |
| 1 | A | A | A | a | −3.06506 | 0.00218 | 71.60% |
| 2 | a | a | a | A | 2.71413 | 0.00664 | 25.00% |
| 3 | a | A | A | a | 0.73651 | 0.46142 | 3% |

‘A’ and ‘a’ in Table 5 indicate alleles. For example, the haplotype No. 1 is a haplotype including four SNPs at which the alleles are ‘A’, ‘A’, ‘A’ and ‘a’. ‘Hap.score’ shows how well the haplotype can classify subjects into the normal group and the disease group. When the ‘p.val’ was 0.05 or less, it was determined that the relationship between the genotype and the disease was significant.

Alternatively, the haplotype for diagnosis myocardial infarction according to an exemplary embodiment may be composed of the polynucleotides of SEQ ID NOS: 241 to 244.

The 101^{st} base, which is the SNP site, of the polynucleotides of SEQ ID NOS: 241 to 244 composing the haplotype can be the risk allele. That is, the haplotype can be a haplotype No. 4 or 7 in Table 6.

TABLE 6

| Haplotype | | | | | | Hap.Freq | y.0 | y.1 |
|---|---|---|---|---|---|---|---|---|
| No. | rs2148582 | rs5050 | rs7079 | rs699 | p-value | total_freq | con_freq | cas_freq |
| 4 | A | A | A | a | 0.024 | 0.110 | 0.135 | 0.088 |
| 5 | A | A | a | a | 0.134 | 0.084 | 0.099 | 0.071 |
| 6 | a | A | a | A | 0.608 | 0.627 | 0.618 | 0.634 |
| 7 | a | a | a | A | 0.035 | 0.172 | 0.142 | 0.198 |

‘Hap.Freq total_freq’ indicates the frequency of the haplotype in the disease group and the normal group.

‘Y.0 con_freq’ indicates the frequency of haplotype in the normal group.

‘Y.1 cas_freq’ indicates the frequency of haplotype in the disease group.

Predicting the incidence of cardiovascular disease using only a genetic factor is difficult since the occurrence of cardiovascular disease may be affected by various environmental or habitual factors. SNPs which are significantly associated with a subject having certain characteristics were selected. In addition, SNPs which exist in the patients with cardiovascular disease but do not exist in the normal persons were identified.

It was that male non-smokers showed a more significant relationship between the existence of the SNPs of a nucleotide sequence of SEQ ID NOS: 57 to 60 and the incidence of myocardial infarction. This was proved by the fact that the male non-smoker had an increased odds ratio in Table 7. Therefore, according to an exemplary embodiment, the SNP for diagnosis of myocardial infarction may be a nucleotide sequence of SEQ ID NOS: 57 to 60 and the subject may be a male non-smoker.

TABLE 7

| | | SEQ ID | | | | |
|---|---|---|---|---|---|---|
| group | alias_id | NO: | OR | OR_LB | OR_UB | Chi_squ_Pvalue |
| Male | MI_1111 | 57 | 1.3816 | 1.0067 | 1.8961 | 0.0939 |
| | MI_1248 | 58 | 1.4353 | 1.0427 | 1.9757 | 0.0674 |
| | MI_2143 | 59 | 1.3513 | 0.98 | 1.8587 | 0.112 |
| | MI_2144 | 60 | 1.4705 | 1.08 | 2 | 0.0547 |
| Male | MI_1111 | 57 | 1.8472 | 1.0138 | 3.3657 | 0.161 |
| smoker | MI_1248 | 58 | 1.8752 | 1.029 | 3.4173 | 0.166 |
| | MI_2143 | 59 | 1.8181 | 1.033 | 3.1746 | 0.122 |
| | MI_2144 | 60 | 1.8518 | 1.0141 | 3.3557 | 0.132 |
| Male | MI_1111 | 57 | 2.2865 | 1.2005 | 4.3546 | 0.0231 |
| non-smoker | MI_1248 | 58 | 2.3976 | 1.2492 | 4.6018 | 0.024 |
| | MI_2143 | 59 | 2.2727 | 1.2004 | 4.3478 | 0.0242 |
| | MI_2144 | 60 | 2.5641 | 1.3458 | 4.7619 | 0.0107 |

The polynucleotide according to an exemplary embodiment may be a nucleotide sequence of SEQ ID NO: 241, 243 or 244 and may be used for the diagnosis of myocardial infarction of a subject having a high C-reactive protein (CRP) level.

The CRP level of blood indicates the degree of inflammation, and is used to measuring the risk of cardiovascular disease. While the CRP level of female subjects in menopause who had cardiovascular disease was 0.42 mg/dl, the CRP level of female subjects in menopause who did not have cardiovascular disease was 0.28 mg/dl (Ridker P M, Hennekens C H, Buring J E, Rifai N., C-reactive protein and other markers of inflammation in the prediction of cardiovascular disease in women, N Engl J Med, 2000, 342:836-843).

In a subject having a polynucleotide of nucleotide sequences of SEQ ID NOS: 241, 243 and 244, the CRP level is not limited since it can be estimated that the subject having a higher CRP level has a relatively higher probability of incidence of cardiovascular disease.

TABLE 8

| | GenBank | | | | |
|---|---|---|---|---|---|
| SEQ | accession | | | | |
| ID | No. of SNP | Risk | Odds | Confidence | |
| NO: | in NCBI | allele | Ratio | interval | Chi_exact_pValue |
| 241 | rs2148582 | G | 0.276 | (0.118, 0.645) | 0.0052 |
| 243 | rs7079 | G | 0.348 | (0.121, 0.999) | 0.0485 |
| 244 | rs699 | G | 3.45 | (1.48, 8.04) | 0.0101 |

The polynucleotide according to an exemplary embodiment may be a nucleotide sequence of SEQ ID NO: 241, 243 or 244 or a complementary polynucleotide thereof, and may be used for the diagnosis of cardiovascular disease of young subjects.

In connection with myocardial infarction, men over 45 and women over 55 who have entered menopause are known to be risk groups.

In a subject having a polynucleotide of nucleotide sequences of SEQ ID NOS: 241, 243 and 244, the age is not limited since it can be estimated that a younger subject has a relatively lower probability of incidence of cardiovascular disease.

TABLE 9

| | GenBank | | | | |
|---|---|---|---|---|---|
| SEQ | accession | | | | |
| ID | No. of SNP | Risk | Odds | Confidence | |
| NO: | in NCBI | allele | Ratio | interval | Chi_exact_pValue |
| 241 | Rs2148582 | G | 0.482 | (0.28, 0.83) | 0.0307 |
| 243 | rs7079 | G | 0.345 | (0.169, 0.706) | 0.0058 |
| 244 | rs699 | G | 2.04 | (1.19, 3.52) | 0.0309 |

The polynucleotide according to an exemplary embodiment may be a nucleotide sequence of SEQ ID NO: 241, 242 or 244 or a complementary polynucleotide thereof, and may be used for the diagnosis of cardiovascular disease of subjects not having diabetes.

Table 10 contains the results obtained from Examples of the present disclosure in which subjects do not have diabetes.

TABLE 10

| | GenBank | | | | |
|---|---|---|---|---|---|
| SEQ | accession | | | | |
| ID | No. of SNP | Risk | Odds | Confidence | |
| NO: | in NCBI | allele | Ratio | interval | Chi_exact_pValue |
| 241 | rs2148582 | G | 0.622 | (0.433, 0.892) | 0.0378 |
| 242 | rs5050 | G | 0.641 | (0.434, 0.946) | 0.0141 |
| 244 | rs699 | G | 1.6 | (1.12, 2.3) | 0.0395 |

The polynucleotide according to an exemplary embodiment may be a nucleotide sequence of SEQ ID NO: 241, 242 or 244 or a complementary polynucleotide thereof, and may be used for the diagnosis of cardiovascular disease of subjects that smoke.

Table 11 contains the results obtained from Examples of the present disclosure in which subjects smoke.

TABLE 11

| | GenBank | | | | |
|---|---|---|---|---|---|
| SEQ | accession | | | | |
| ID | No. of SNP | Risk | Odds | Confidence | |
| NO: | in NCBI | allele | Ratio | interval | Chi_exact_pValue |
| 241 | rs2148582 | G | 0.545 | (0.317, 0.938) | 0.0392 |
| 242 | rs5050 | G | 0.465 | (0.236, 0.914) | 0.0458 |
| 244 | rs699 | G | 1.83 | (1.07, 3.16) | 0.0392 |

The polynucleotide according to an exemplary embodiment may be SEQ ID NO: 243 or a complementary polynucleotide thereof, and may be used for the diagnosis of cardiovascular disease of subjects having a high TG level.

As a result of an 8-year PROCAM study, it was discovered that the TG level during an empty stomach is quantitatively relative to the frequency of cardiovascular disease. When a subject had a quite high TG level, for example 400-799 mg/dl, it was found that the incidence rate of the cardiovascular disease increased more than three times relative to the normal TG level.

In a subject having the polynucleotide of SEQ ID NO: 243, the TG level is not limited since a subject showing a high TG level has a relatively higher probability of incidence of cardiovascular disease.

TABLE 12

| | GenBank | | | | |
|---|---|---|---|---|---|
| SEQ | accession | | | | |
| ID | No. of SNP | Risk | Odds | Confidence | |
| NO: | in NCBI | allele | Ratio | interval | Chi_exact_pValue |
| 243 | rs7079 | G | 0.167 | (0.0341, 0.814) | 0.035 |

In an exemplary embodiment, a polynucleotide containing a SNP may include at least 8 contiguous nucleotides, for example, 8 to 70 contiguous nucleotides.

In an aspect of the present disclosure, a polynucleotide is specifically hybridized with a polynucleotide of SEQ ID NOS: 1 to 60 and 241 to 244 or a complementary polynucleotide thereof. The polynucleotide may be allele-specific.

The polynucleotide may include at least 8 contiguous nucleotides of these sequences, for example, 8 to 70 contiguous nucleotides.

The allele-specific polynucleotide is a polynucleotide specifically hybridized with each allele base of the polynucleotide. The hybridization can be performed to specifically distinguish the bases in polymorphic sites among polymorphic sequences of SEQ ID NOS: 1 to 60 and 241 to 244. The hybridization can be carried out under strict conditions, for example, in a salt concentration of 1 M or less and at a temperature of 25° C. or higher. For example, 5×SSPE (750 mM NaCl, 50 mM Na Phosphate, 5 mM EDTA, pH 7.4) and 25 to 30° C. may be suitable conditions for the allele-specific probe hybridization.

In an exemplary embodiment, the allele-specific polynucleotide can be a primer. The primer is a single-strand oligonucleotide capable of initiating template-directed DNA synthesis in an appropriate buffer under appropriate conditions, for example, in the presence of four different nucleotide triphosphates and a polymerizing agent such as DNA, RNA polymerase or reverse transcriptase at a proper temperature. The length of the primer may vary according to the purpose of use, but is 15 to 30 nucleotides in an exemplary embodiment. A short primer molecule can require a lower temperature to be stably hybridized with the template. The primer sequence does not necessarily need to be completely complementary to the template, but can be sufficiently complementary to be hybridized with the template. The 3′ end of the primer is arranged to correspond to the polymorphic sites of SEQ ID NOS: 1 to 60 and 241 to 244. The primer is hybridized with the target DNA including the polymorphic site and initiates amplification of an allele having complete homology to the primer. The primer and another primer hybridized with the other side are used as a primer pair. Amplification is performed from the two primers, indicating that there is a specific allele in the polynucleotide. According to an exemplary embodiment, the primer includes a polynucleotide fragment used in a ligase chain reaction (LCR). For example, the primer may be a polynucleotide used in the Examples below.

In an exemplary embodiment, an allele specific polynucleotide may be a probe. The probe is a hybridization probe, which is an oligonucleotide capable of binding specifically to a complementary strand of a nucleic acid. Such a probe includes a peptide nucleic acid introduced by Nielsen et al., Science 254, 1497-1500 (1991). According to an exemplary embodiment, the probe is an allele-specific probe. When a polymorphic site is located in nucleic acid fragments derived from two members of the same species, the allele-specific probe can hybridize with the DNA fragment derived from one member but not with the DNA fragment derived from the other member. In this case, the hybridization conditions can be suitable for hybridization with only one allele by facilitating a significant difference in intensities of hybridization for different alleles. According to an exemplary embodiment, the probe is arranged such that its central site is the polymorphic site of the sequence, for example the 7^{th} position in a probe consisting of 15 nucleotides, or the 8^{th} or 9^{th} position in a probe consisting of 16 nucleotides. In this way, a difference in hybridization for different alleles can be obtained. According to an exemplary embodiment, the probe can be used in a diagnosis method for detecting an allele, etc. The diagnosis method may be Southern blotting in which detection is performed using the hybridization of nucleic acids, or a method in which a microarray to which the probe is bound in advance is used.

A polypeptide according to an aspect of the present disclosure is encoded by a polynucleotide of SEQ ID NOS: 1 to 60 and 241 to 244.

Particularly, the amino acid of the polypeptide encoded by the polynucleotide of SEQ ID NO: 56 including MI_{—}1503 located in the CYBA gene changed. The amino acid of the polypeptide encoded by the polynucleotide of SEQ ID NO: 50 including MI_{—}1264 located in the MST1R gene and the polynucleotide of SEQ ID NO: 58 including MI_{—}1248 located in the polymerase iota gene also changed. The sites at which the three amino acids changed are respectively the 72^{nd} amino acid of the CYBA protein, the 1335^{th} amino acid of the MST1R protein and the 706^{th} amino acid of the polymerase iota protein.

An antibody according to an aspect of the present disclosure specifically binds to a polypeptide encoded by a polynucleotide of SEQ ID NOS: 1 to 60 and 241 to 244. The antibody may be a monoclonal antibody.

A microarray according to an aspect of the present disclosure includes one or more polynucleotides of a nucleotide sequence of SEQ ID NOS: 1 to 60 and 241 to 244 and complementary polynucleotides thereof, one or more polynucleotides capable of specifically hybridizing with the polynucleotides of SEQ ID NOS: 1 to 60 and 241 to 244, one or more polypeptides encoded by one of the polynucleotides, or one or more cDNAs thereof.

The microarray may be prepared using a conventional method known to those skilled in the art using the polynucleotides, probes or polynucleotides that hybridize with the probe, the polypeptide encoded by one of the polynucleotides or cDNA thereof.

For example, the polynucleotide may be fixed to a substrate coated with an active group such as amino-silane, poly-L-lysine or aldehyde. Also, the substrate may be composed of silicon, glass, quartz, metal or plastic or other suitable materials. A polynucleotide may be fixed to the substrate by micropipetting using a piezoelectric method or by using a spotter in the shape of a pin, or any suitable technique.

A kit according to an aspect of the present disclosure includes a polynucleotide of a nucleotide sequence of SEQ ID NOS: 1 to 60 and 241 to 244, a polynucleotide hybridized with one of the polynucleotides, a polypeptide encoded by one of the polynucleotides or cDNA thereof.

The kit may further include a primer set used for isolating DNA including SNPs from diagnosed subjects and amplifying the DNA. The appropriate primer set may be determined by those skilled in the art. For example, the primer set in Examples of the present disclosure may be used. Also, the kit may further include a reagent for a polymerizing reaction, for example dNTP, various polymerases and colorants.

An identifying method according to an aspect of the present disclosure includes using the SNP to identify a subject having a changed risk of incidence of myocardial infarction.

An identifying method can include isolating a nucleic acid sample from a subject and determining an allele at polymorphic sites of one or more polynucleotides among SEQ ID NOS: 1 to 60 and 241 to 244, wherein the polymorphic sites are positioned at the 101^{st} nucleotides of the polynucleotides.

In an exemplary embodiment, the isolation of the DNA from the subject can be carried out by performing a method known to those skilled in the art. For example, DNA can be directly purified from tissues or cells or a specific region can be amplified using a polymerase chain reaction (PCR), etc. and isolated. In the detailed description, DNA refers not only to DNA, but also to cDNA synthesized from mRNA. Nucleic acids can be obtained from a subject using PCR amplification, ligase chain reaction (LCR) (Wu and Wallace, Genomics 4, 560 (1989), Landegren, etc., Science 241, 1077 (1988)), transcription amplification (Kwoh, etc., Proc. Natl. Acad. Sci. USA 86, 1173 (1989)), self-sustained sequence replication (Guatelli, etc., Proc. Natl. Acad. Sci. USA 87, 1874 (1990)) or Nucleic Acid Sequence Based Amplification (NASBA).

Sequencing of the isolated DNA may be performed through various methods known to those skilled in the art. For example, the nucleotides of nucleic acids may be directly sequenced using a dideoxy method. Also, the nucleotides of the polymorphic sites may be sequenced by hybridizing the DNA with a probe containing the sequence of the SNP site and a complementary probe thereof, and examining the degree of the hybridization. The degree of hybridization may be measured using a method of indicating a detectable index of the target DNA and specifically detecting the hybridized target, or using an electrical signal detecting method.

Particularly, the sequencing may be carried out using allele-specific probe hybridization, allele-specific amplification, sequencing, 5′ nuclease digestion, molecular beacon assay, oligonucleotide ligation assay, size analysis or single-stranded conformation polymorphism method.

In an exemplary embodiment, the method of diagnosing myocardial infarction may further include judging that the subject has an increased risk of incidence of myocardial infarction when an allele at a polymorphic site of one or more polynucleotides of SEQ ID NOS: 1 to 60 and 241 to 244 is a risk allele.

According to an exemplary embodiment, the risk allele is determined based on the allele ‘A’. When a frequency of the allele ‘A’ in the disease group is higher than in the normal group, ‘A’ is regarded as a risk allele. In the opposite case, ‘a’ is regarded as a risk allele. subjects having more risk alleles have a higher probability of having myocardial infarction.

In an exemplary embodiment, the risk may be an increased risk or a decreased risk. When the frequency of an allele is higher in the normal group than in the disease group, the risk to a subject with the allele may be decreased. On the other hand, when the frequency of an allele of the SNP is higher in the disease group than in the normal group, the risk to a subject with the allele can be increased.

In a method of diagnosis of myocardial infarction according to an exemplary embodiment, the subject may be male and does not smoke and the polynucleotide determining the allele at the polymorphic site in the subject may be a nucleotide sequence of SEQ ID NOS: 57 to 60.

Also, the subject may have a high CRP level and the polynucleotide determining the allele at the polymorphic site in the subject may be a nucleotide sequence of SEQ ID NO: 241, 243 or 244.

The subject may be young and the polynucleotide determining the allele at the polymorphic site in the subject may be a nucleotide sequence of SEQ ID NO: 241, 243 or 244.

The subject may not have diabetes and the polynucleotide determining the allele at the polymorphic site in the subject may be a nucleotide sequence of SEQ ID NO: 241, 243 or 244.

The subject may smoke and the polynucleotide determining the allele at the polymorphic site in the subject may be a nucleotide sequence of SEQ ID NO: 241, 242 or 244.

The subject may have a high TG level and the polynucleotide determining the allele at the polymorphic site in the subject may be SEQ ID NO: 243.

In a method of diagnosing myocardial infarction according to an exemplary embodiment, a haplotype may be used. The polynucleotide may consist of SEQ ID NOS: 57 to 60. Alternatively, the polynucleotide may consist of SEQ ID NOS: 241 to 244.

A method of detecting SNPs in nucleic acid molecules according to an aspect of the present disclosure includes contacting a test sample containing nucleic acid molecules with a reagent specifically hybridized under strict conditions with a polynucleotide of a nucleotide sequence of SEQ ID NOS: 1 to 60 and 241 to 244 containing at least 8 contiguous nucleotides and the 101^{st} base of the nucleotide sequence and complementary polynucleotides of the nucleotide sequences, and detecting the formation of a hybridized double-strand.

The detecting of the formation of a hybridized double-strand is carried out using allele-specific probe hybridization, allele-specific amplification, sequencing, 5′ nuclease digestion, molecular beacon assay, oligonucleotide ligation assay, size analysis or single-stranded conformation polymorphism.

A method of screening pharmaceutical compositions for myocardial infarction according to an aspect of the present disclosure includes contacting a candidate material with a polypeptide encoded by a polynucleotide of a nucleotide sequence of SEQ ID NOS: 1 to 60 and 241 to 244 containing at least 8 contiguous nucleotides and the 101^{st} base of the nucleotide sequence and complementary polynucleotides of the nucleotide sequences under proper conditions for the formation of a binding complex, and detecting the formation of the binding complex from the polypeptide and the candidate material.

Detecting the formation of the binding complex may be carried out through coimmunoprecipitation, Radioimmunoassay (RIA), Enzyme Linked ImmunoSorbent Assay (ELISA), Immunohistochemistry, Western Blotting or Fluorescence Activated Cell Sorer (FACS).

A method of regulating gene expression according to an exemplary embodiment includes binding an anti-sense nucleotide or Si RNA with a polynucleotide of nucleotide sequence of SEQ ID NOS: 1 to 60 and 241 to 244 containing at least 8 contiguous nucleotides and the 101^{st} base of the nucleotide sequence and complementary polynucleotides of the nucleotide sequences, wherein the anti-sense nucleotide and Si RNA are specific to the polynucleotide.

The present disclosure will now be described in greater detail with reference to the following examples. The following examples are for illustrative purposes only and are not intended to limit the scope of the disclosure.

### EXAMPLE 1

SNP Selection

DNA was isolated from blood of a disease group diagnosed with a cardiovascular disease and treated, and DNA was isolated from a normal group not having symptoms of cardiovascular disease, and then an appearance frequency of a specific SNP was analyzed. Both groups consisted of Koreans. The SNP of the Examples of the present disclosure was selected from either a published database (NCBI dbSNP:http://www.ncbi.nlm.nih.gov/SNP/) or a Sequenom website (http://www.realsnp.com/). The SNPs were analyzed using a primer close to the selected SNP.

1-1. Preparation of DNA Sample

DNA was extracted from blood of a disease group consisting of 221 Korean male patients diagnosed with myocardial infarction and DNA was extracted from a normal group consisting of 192 Korean men not having myocardial infarction symptoms. The chromosome DNA extraction was carried out according to a known method (Molecular cloning: A Laboratory Manual, p 392, Sambrook, Fritsch and Maniatis, 2nd edition, Cold Spring Harbor Press, 1989) and the guidelines of a commercially available kit (Gentra system, D-50K). Only DNA having a purity of at least 1.7 measured using UV light (260/280 nm) was selected from the extracted DNA and used.

1-2. Amplification of the Target DNA

Target DNA containing a certain DNA region including SNPs to be analyzed was amplified using a PCR. The PCR was performed using a conventional method and the conditions were as indicated below. 2.5 ng/ml of target genome DNA was first prepared. Then the following PCR reaction solution was prepared.

| | |
|---|---|
| Water (HPLC grade) | 3.14 μl |
| 10×buffer | 0.5 μl |
| MgCl₂ 25 mM | 0.2 μl |
| dNTP mix (GIBCO)(25 mM/each) | 0.04 μl |
| Taq pol (HotStart)(5 U/μl) | 0.02 μl |
| Forward/reverse primer mix (10 μM) | 0.1 μl |
| DNA | 1.00 μl |
| Total volume | 5.00 μl |

Here, the forward and reverse primers were selected upstream and downstream from the SNP at proper positions. The primer set is listed in Table 13.

Thermal cycling of a PCR was performed by maintaining the temperature at 95° for 15 minutes, cycling the temperature from 95° for 30 seconds, to 56° for 30 seconds, to 72° for 1 minute a total of 45 times, maintaining the temperature at 72° for 3 minutes, and then storing at 4°. All temperatures are given in Celsius units unless indicated otherwise.

As a result, target DNA fragments having 200 nucleotides or less were obtained.

1-3. Analysis of SNP of the Amplified Target DNA

Analysis of the SNPs in the target DNA fragments was performed using a homogeneous Mass Extend (hME) technique from Sequenom. The principle of the hME technique is as follows. First, a primer, also called an extension primer, complementary to bases up to just before the SNP of the target DNA fragment was prepared. Next, the primer was hybridized with the target DNA fragment and DNA polymerization was facilitated. At this time, a reagent (Termination mix, e.g. ddTTP) for terminating the polymerization was added to the reaction solution after the base complementary was added to a first allele base (e.g. ‘A’ allele) among the subject SNP alleles. As a result, when the target DNA fragment included the first allele (e.g. ‘A’ allele), a product having only one base complementary to the added first allele (e.g. ‘T’) was obtained. On the other hand, when the target DNA fragment included a second allele (e.g. ‘G’ allele), a product having a base complementary to the second allele (e.g. ‘C’) and extending to the first allele base (e.g. ‘A’) was obtained. The length of the product extending from the primer was determined using mass analysis to determine the type of allele in the target DNA. Specific experimental conditions were as follows.

First, free dNTPs were removed from the PCR product. To this end, 1.53 μl of pure water, 0.17 μl of a hME buffer and 0.30 μl of shrimp alkaline phosphatase (SAP) were added to a 1.5 ml tube and mixed to prepare a SAP enzyme solution. The tube was centrifuged at 5,000 rpm for 10 seconds. Then, the PCR product was put into the SAP solution tube, sealed, maintained at 37° for 20 minutes and at 85° for 5 minutes, and then stored at 4°.

Next, homogeneous extension was performed using the target DNA product as a template. The reaction solution was as follows.

| | |
|---|---|
| Water (nanopure grade) | 1.728 μl |
| hME extension mix (10×buffer containing 2.25 mM d/ddNTPs) | 0.200 μl |
| Extension primer (each 100 μM) | 0.054 μl |
| Thermosequenase (32 U/μl) | 0.018 μl |
| Total volume | 2.00 μl |

The reaction solution was mixed well and spin down centrifuged. A tube or plate containing the reaction solution was sealed, maintained at 94° for 2 minutes, cycled from 94° for 5 seconds, to 52° for 5 seconds, to 72° for 5 seconds a total of 40 times, and then stored at 4°. The obtained homogeneous extension product was washed with a resin (SpectroCLEAN, Sequenom, #10053) and a salt was removed. The extension primers used for homogeneous extension are indicated in Table 13.

TABLE 13

| Nucleotide containing | Primer for target DNA | | Extension |
|---|---|---|---|
| SNP | amplification (SEQ ID NO:) | | primer |
| (SEQ ID NO:) | Forward primer | Reverse primer | (SEQ ID NO:) |
| 1 | 61 | 62 | 63 |
| 2 | 64 | 65 | 66 |
| 3 | 67 | 68 | 69 |
| 4 | 70 | 71 | 72 |
| 5 | 73 | 74 | 75 |
| 6 | 76 | 77 | 78 |
| 7 | 79 | 80 | 81 |
| 8 | 82 | 83 | 84 |
| 9 | 85 | 86 | 87 |
| 10 | 88 | 89 | 90 |
| 11 | 91 | 92 | 93 |
| 12 | 94 | 95 | 96 |
| 13 | 97 | 98 | 99 |
| 14 | 100 | 101 | 102 |
| 15 | 103 | 104 | 105 |
| 16 | 106 | 107 | 108 |
| 17 | 109 | 110 | 111 |
| 18 | 112 | 113 | 114 |
| 19 | 115 | 116 | 117 |
| 20 | 118 | 119 | 120 |
| 21 | 121 | 122 | 123 |
| 22 | 124 | 125 | 126 |
| 23 | 127 | 128 | 129 |
| 24 | 130 | 131 | 132 |
| 25 | 133 | 134 | 135 |
| 26 | 136 | 137 | 138 |
| 27 | 139 | 140 | 141 |
| 28 | 142 | 143 | 144 |
| 29 | 145 | 146 | 147 |
| 30 | 148 | 149 | 150 |
| 31 | 151 | 152 | 153 |
| 32 | 154 | 155 | 156 |
| 33 | 157 | 158 | 159 |
| 34 | 160 | 161 | 162 |
| 35 | 163 | 164 | 165 |
| 36 | 166 | 167 | 168 |
| 37 | 169 | 170 | 171 |
| 38 | 172 | 173 | 174 |
| 39 | 175 | 176 | 177 |
| 40 | 178 | 179 | 180 |
| 41 | 181 | 182 | 183 |
| 42 | 184 | 185 | 186 |
| 43 | 187 | 188 | 189 |
| 44 | 190 | 191 | 192 |
| 45 | 193 | 194 | 195 |
| 46 | 196 | 197 | 198 |
| 47 | 199 | 200 | 201 |
| 48 | 202 | 203 | 204 |
| 49 | 205 | 206 | 207 |
| 50 | 208 | 209 | 210 |
| 51 | 211 | 212 | 213 |
| 52 | 214 | 215 | 216 |
| 53 | 217 | 218 | 219 |
| 54 | 220 | 221 | 222 |
| 55 | 223 | 224 | 225 |
| 56 | 226 | 227 | 228 |
| 57 | 229 | 230 | 231 |
| 58 | 232 | 233 | 234 |
| 59 | 235 | 236 | 237 |
| 60 | 238 | 239 | 240 |
| 241 | 245 | 246 | 247 |
| 242 | 248 | 249 | 250 |
| 243 | 251 | 252 | 253 |
| 244 | 254 | 255 | 256 |

Mass analysis was performed on the obtained extension product to determine sequence of a polymorphic site using Matrix Assisted Laser Desorption and Ionization-Time of Flight (MALDI-TOF). In the MALDI-TOF, a material to be analyzed was exposed to laser beam, and flew with an ionized matrix (e.g. 3-Hydroxypicolinic acid) in a vacuum to a detector. The flying time to the detector was calculated to determine the mass. A light material could reach the detector in a shorter amount of time than a heavy material. The nucleotide sequences of SNPs in the target DNA may be determined based on differences in mass and known nucleotide sequences of the SNPs.

1-4. Selection of SNP

Allele frequencies in the disease group consisting of 221 Korean male patients diagnosed with myocardial infarction and treated and allele frequencies of the normal group consisting of 192 Korean men not having symptoms of myocardial infarction were compared. The Fisher's exact test was performed based on the frequency as an association test.

The effect size was assumed using an allele odds ratio at a 95% confidence interval. When the normal group had a higher frequency of a given allele than the disease group, the allele was determined to be associated with a decreased risk of myocardial infarction and the other allele was determined to be the risk allele of myocardial infarction. On the other hand, when the disease group had a higher frequency of a given allele than the normal group, the allele was determined to be the risk allele.

In the allele association test of a SNP, when the p-value was 0.05 or less, the SNP was regarded as a noticeable genetic marker.

The results are listed in Tables 1 to 3. As indicated in Tables 1 to 3, 64 SNPs associated with myocardial infarction were identified.

1-5. Selection of Haplotype

Four SNPs of SEQ ID NOS: 57 to 69 in a polymerase iota gene which is associated with DNA repair are disclosed in Table 5. In addition, four SNPs of SEQ ID NOS: 241 to 244 are disclosed in Table 6.

1-6. Investigation into Environmental or Habitual Factor Dependence of SNP

The disease group consisting of 221 Korean male patients diagnosed with myocardial infarction and treated and the normal group consisting of 192 Korean men not having symptoms of myocardial infarction were respectively divided into subgroups in consideration of a degree of risk in connection with environmental or habitual factors associated with myocardial infarction, and allele frequencies were compared. The results were shown in Tables 7 to 12.

### EXAMPLE 2

Preparation of SNP Immobilized Microarray

A microarray was prepared by immobilizing the selected SNPs on a substrate. That is, polynucleotides of nucleotide sequences of SEQ ID NOS: 1 to 60 and 241 to 244 including 20 contiguous nucleotides and 101^{st} base of the nucleotide sequence were immobilized on the substrate, wherein each SNP was located at the 11^{th} of the 20 nucleotides.

First, N-ends of each of the polynucleotides were substituted with an amine group and the polynucleotides were spotted onto a silylated slide (Telechem) where 2×SSC (pH 7.0) of a spotting buffer was used. After spotting, binding was induced in a drying machine and free oligonucleotides were removed by washing with a 0.2% SDS solution for 2 minutes and with triple distilled water for 2 minutes. The microarray was prepared using denaturation induced by increasing the temperature of the slide to 95° C. for 2 minutes, washing with a blocking solution (1.0 g NaBH₄, PBS (pH 7.4) 300 mL, EtOH 100 mL) for 15 minutes, a 0.2% SDS solution for 1 minute and triple distilled water for 2 minutes, and then drying at room temperature.

### EXAMPLE 3

Diagnosis of Myocardiar Infarction Using the Microarray

A target DNA was isolated from blood of a subject to diagnose the incidence or possibility of myocardial infarction and was labeled with a fluorescent material using the methods described in Examples 1-1 and 1-2. The fluorescent labeled target DNA was hybridized with the microarray prepared in Example 2 at 42° C. for 4 hours in a UniHyb hybridization solution (TeleChem). The slide was washed twice with 2×SSC at room temperature for 5 minutes and dried in air. The dried slide was scanned using a ScanArray 5000 (GSI Lumonics). The scanned results were analyzed using a QuantArray (GSI Lumonics) and an ImaGene software (BioDiscover). The probability of incidence of myocardial infarction and the susceptibility thereto were measured by identifying whether the subject had the SNP according to an exemplary embodiment.

The SNP and haplotype associated with myocardial infarction according to the present disclosure may be used for diagnosis and treatment of myocardial infarction and gene fingerprint analysis. By using the microarray and the kit including the SNP of the present disclosure, myocardial infarction can be effectively diagnosed. According to the method of analyzing SNPs associated with myocardial infarction of the present disclosure, the presence or risk of myocardial infarction can effectively be diagnosed.

While the present disclosure has been particularly shown and described with reference to exemplary embodiments thereof, it will be understood by those of ordinary skill in the art that various changes in form and details may be made therein without departing from the spirit and scope of the present disclosure as defined by the following claims.

What is claimed is:

1. A polynucleotide comprising at least 8 contiguous nucleotides that include the 101^{st} base of a nucleotide sequence selected from the group consisting of nucleotide sequences of SEQ ID NOS: 1 to 60 and 241 to 244 and complementary nucleotide sequences.

2. The polynucleotide of claim 1 wherein the nucleotide sequence is selected from among SEQ ID NOS: 57 to 60.

3. The polynucleotide of claim 1 wherein the nucleotide sequence is selected from among SEQ ID NOS: 241 to 244.

4. A polynucleotide for the diagnosis of myocardial infarction in a subject who is male and does not smoke comprising a polynucleotide of claim 1 wherein the nucleotide sequence is selected from among SEQ ID NOS: 57 to 60.

5. A polynucleotide for the diagnosis of myocardial infarction in a subject who has a high C-reactive protein (CRP) level comprising a polynucleotide of claim 1 wherein the nucleotide sequence is selected from among SEQ ID NOS: 241, 243 and 244.

6. A polynucleotide for the diagnosis of myocardial infarction in a young subject comprising a polynucleotide of claim 1 wherein the nucleotide sequence is selected from among SEQ ID NOS: 241, 243 and 244.

7. The polynucleotide of claim 6, wherein the age of the young subject is 55 or less.

8. A polynucleotide for the diagnosis of myocardial infarction in a subject without diabetes comprising a polynucleotide of claim 1 wherein the nucleotide sequence is selected from among SEQ ID NOS: 241, 242 and 244.

9. A polynucleotide for the diagnosis of myocardial infarction in a subject who smokes comprising a polynucleotide of claim 1 wherein the nucleotide sequence is selected from among SEQ ID NOS: 241, 242 and 244.

10. A polynucleotide for the diagnosis of myocardial infarction in a subject who has a high triglycerol (TG) level comprising a polynucleotide of claim 1 wherein the selected nucleotide sequence is SEQ ID NO: 243.

11. The polynucleotide of claim 1 comprising 8 to 70 contiguous nucleotides.

12. A polynucleotide capable of specifically hybridizing with the polynucleotide of claim 1.

13. The polynucleotide of claim 12 comprising 8 to 70 contiguous nucleotides.

14. An allele specific probe comprising a polynucleotide of claim 12.

15. An allele specific primer comprising a polynucleotide of claim 12.

16. A polypeptide encoded by a polynucleotide of claim 1.

17. An antibody capable of specifically binding to the polypeptide of claim 16.

18. The antibody of claim 17 wherein the antibody is a monoclonal antibody.

19. A microarray for detecting a SNP comprising:
a polynucleotide of claim 1 or a polynucleotide capable of specifically hybridizing with the polynucleotide of claim 1;
a polypeptide encoded by a polynucleotide of claim 1 or a polynucleotide capable of specifically hybridizing with the polynucleotide of claim 1; or
a cDNA of a polynucleotide of claim 1 or a polynucleotide capable of specifically hybridizing with the polynucleotide of claim 1.

20. A kit for detecting a SNP comprising:
a polynucleotide of claim 1 or a polynucleotide capable of specifically hybridizing with the polynucleotide of claim 1;
a polypeptide encoded by a polynucleotides of claim 1 or a polynucleotide capable of specifically hybridizing with the polynucleotide of claim 1; or
a cDNA of a polynucleotide of claim 1 or a polynucleotide capable of specifically hybridizing with the polynucleotide of claim 1.

21. A method of identifying a subject having a changed risk of incidence of myocardial infarction, the method comprising:
isolating a nucleic acid sample from the subject; and
determining an allele at a polymorphic site of one or more polynucleotides selected from the group consisting of nucleotide sequences of SEQ ID NOS: 1 to 60 and 241 to 244, wherein the polymorphic site is positioned at the 101^{st} nucleotide of the polynucleotides.

22. The method of claim 21,
wherein determining the allele is carried out by performing a method selected from the group consisting of allele-specific probe hybridization, allele-specific amplification, sequencing, 5′ nuclease digestion, molecular beacon assay, oligonucleotide ligation assay, size analysis and single-stranded conformation polymorphism.

23. The method of claim 21, wherein the changed risk is an increased risk.

24. The method of claim 21, wherein the changed risk is a decreased risk.

25. The method of claim 21 further comprising
judging that the subject has an increased risk of incidence of myocardial infarction when an allele at a polymorphic site of one or more polynucleotides selected from the group consisting of nucleotide sequences of SEQ ID NOS: 1 to 60 and 241 to 244 is a risk allele.

26. The method of claim 21, wherein the subject is male and does not smoke and the polynucleotide is selected from the group consisting of nucleotide sequences of SEQ ID NOS: 57 to.

27. The method of claim 21, wherein the subject has a high CRP level and the polynucleotide is selected from the group consisting of nucleotide sequences of SEQ ID NOS: 241, 243 and 244.

28. The method of claim 21, wherein the subject is young and the polynucleotide is selected from the group consisting of nucleotide sequences of SEQ ID NOS: 241, 243 and 244.

29. The method of claim 21, wherein the subject does not have diabetes and the polynucleotide is selected from the group consisting of nucleotide sequences of SEQ ID NOS: 241, 242 and 244.

30. The method of claim 21, wherein the subject smokes and the polynucleotide is selected from the group consisting of nucleotide sequences of SEQ ID NOS: 241, 242 and 244.

31. The method of claim 21, wherein the subject has a high triglycerol (TG) level and the polynucleotide is SEQ ID NO: 243.

32. The method of claim 21, wherein the polynucleotide is consisting of nucleotide sequences of SEQ ID NOS: 57 to 60.

33. The method of claim 21, wherein the polynucleotide is consisting of nucleotide sequences of SEQ ID NOS: 241 to 244.

34. A method of detecting a SNP in nucleic acid molecules, the method comprising:
contacting a test sample containing nucleic acid molecules with a reagent capable of specifically hybridizing under strict conditions with a polynucleotide selected from the group consisting of nucleotide sequences of SEQ ID NOS: 1 to 60 and 241 to 244 comprising at least 8 contiguous nucleotides and the 101^{st} base of the nucleotide sequence and complementary polynucleotides of the nucleotide sequences; and
detecting the formation of a hybridized double-strand.

35. The method of claim 34,
wherein detecting the formation of a hybridized double-strand is carried out by performing a method selected form the group consisting of allele-specific probe hybridization, allele-specific amplification, sequencing, 5′ nuclease digestion, molecular beacon assay, oligonucleotide ligation assay, size analysis and single-stranded conformation polymorphism.

36. A method of screening pharmaceutical compositions for effect on myocardial infarction, the method comprising:
contacting a candidate material with a polypeptide encoded by a polynucleotide selected from the group consisting of nucleotide sequences of SEQ ID NOS: 1 to 60 and 241 to 244 comprising at least 8 contiguous nucleotides and the 101^{st} base of the nucleotide sequence and complementary polynucleotides of the nucleotide sequences under proper conditions for the formation of a binding complex; and
detecting the formation of a binding complex of the polypeptide and the candidate material.

37. The method of claim 36, wherein detecting the formation of the binding complex is carried out by performing a method selected from the group consisting of coimmunoprecipitation, Radioimmunoassay (RIA), Enzyme Linked ImmunoSorbent Assay (ELISA), Immunohistochemistry, Western Blotting and Fluorescence Activated Cell Sorer (FACS).

38. A method of regulating gene expression, the method comprising
binding an anti-sense nucleotide or Si RNA with a polynucleotide comprising at least 8 contiguous nucleotides and the 101^{st} base of a nucleotide sequence selected from the group consisting of nucleotide sequences of SEQ ID NOS: 1 to 60 and 241 to 244 and complementary polynucleotide thereof, wherein the anti-sense nucleotide and Si RNA are specific to the polynucleotide.
